(19) United States
(12) Patent Application Publication
(10) Pub. No.: US 2011/0117068 A1
(43) Pub. Date: May 19, 2011

(54) PROBIOTIC MICROORGANISMS FOR THE REDUCTION OF MANURE ODOR

(75) Inventors: Christine Lang, Berlin (DE); Stefanie Arya, Berlin (DE); Natalia Bolotina, Berlin (DE); Markus Veen, Altmühldorf (DE); Mewes Böttner, Heidelberg (DE); Eckhard Budde, Köln (DE); Andreas Künkel, Speyer (DE); Angelika-Maria Pfeiffer, Lambrecht (DE)

(73) Assignee: OrganoBalance GmbH, Berlin (DE)

(21) Appl. No.: 12/377,712

(22) PCT Filed: Aug. 20, 2007
(86) PCT No.: PCT/EP07/07337

§ 371 Date: Nov. 8, 2010

(30) Foreign Application Priority Data
Aug. 18, 2006 (EP) 06017243.4

(51) Int. Cl. A61K 35/74; C12N 1/20; C12N 1/16; A61P 1/14; A61K 36/062; A61K 36/06

(52) U.S. Cl. 424/93.45; 435/252.9; 435/255.1; 435/255.3; 435/255.4; 424/93.51; 424/195.16; 424/780

(57) ABSTRACT

Described are microorganism which are able to reduce the generation of feces odor by decreasing the amount of at least one of the compounds methyl mercaptan, a sulphide compound, cadavarine, putrescine, indole or skatole, and wherein said decrease in the amount of said compounds is independent of the growth of the microorganism. Also described are compositions, comprising such microorganisms, e.g. food, feed or pharmaceutical compositions and the use of such microorganisms for suppressing feces odor or the preparation of foodstuff or feedstuff, as well as corresponding methods for the production of food or feed composition and additives for food, feed or drinks.

The present invention relates to microorganisms which are able to reduce the generation of feces odor by decreasing the amount of at least one of the compounds, methyl mercaptan, a sulphide compound, cadavarine, putrescine, indole or skatole, and wherein said decrease in the amount of said compounds is independent of the growth of the microorganism. The present invention also relates to compositions, comprising such microorganisms, e.g. food, feed or pharmaceutical compositions and the use of such microorganisms for suppressing feces odor or the preparation of foodstuff or feedstuff. A corresponding method for the production of food or feed composition and additives for food, feed or drinks comprising such microorganisms are also provided by the present invention.

The expansion of the livestock industry has caused general public concern about the potential impact of intensive animal operations on the environment. Odor is one of the greatest concerns to the public when considering the siting of new or the expansion of existing livestock operations. Feces odor is produced by the incomplete anaerobic breakdown of feed components, especially peptides, in the large intestine (Burnett, 1969, Miner, 1977, Ritter, 1989). Microorganisms play a central role in the production of these odors as they carry out anaerobic fermentation of substrates in the intestine. The result of this incomplete fermentation is a complex mixture of malodorous substances (Mackie et al. 1998). Substances, which are predominantly mentioned as compounds mainly correlating with feces odor are mercaptan, hydrogen sulphide, volatile fatty acids, skatole, indole and biogenic amines.

The issue, which substances in feces do most significantly contribute to odor, has been controversially discussed in the prior art. From 168 compounds identified in livestock waste, 30 had an odor detection threshold of less than 1 ppm (O'Neill and Phillips, 1992), making them candidates for odor contribution. Sulphur compounds were regarded as important by Fakhoury et al. (2000), who identified hydrogen sulphide as having the highest correlation with malodor. Sulphur compounds in general were identified as being the major class contributing to malodor and hydrogen sulphide being the most important single substance mentioned by Suarez et al. (1998). Moore et al. (1987) identified methyl sulphur compounds (mercaptans) as a major class of odorous substances contributing to feces odor. Indole was found to be one of the most highly correlating substances by Schaefer (1977) as well as by Yashahura (1987), who in addition found skatole to be comparably important. Volatile fatty acids were also found to correlate with odor intensity by Schaefer (1997). Sato et al. (2001) found volatile fatty acids to account for 90% of malodorous substances in feces and therefore suggests those to be the major contributors to odor. Also biogenic amines like cadaverine and putrescine are important contributors to malodor (Tabor and Tabor, 1985).

Another important aspect is the observation that pure odorants can be accurately analysed with respect to concentration, odor quality and odor threshold, whereas the individual impact on the entire odor perception remained difficult to evaluate. Under conditions when compounds as mercaptans, hydrogen sulphide or other typical compounds are detectable in a mixture by a human taster, the measured concentration of the single compound in the air can still be lower than the detectable odor threshold. This effect is known as “synergist action of odorants”. As a consequence a good correlation between sensory and chemical analysis cannot be achieved (Fakhoury et al. 2000).

Concern about air pollution from livestock operations has led to more research into method development for the reduction and control of odors. Masking agents, enzymes and bacterial preparations, feed additives, diet modifications, chemicals, oxidation processes, air scrubbers, biofilters and new ventilation systems have been developed and studied. Masking, desinfecting and oxidizing agents can provide short-term control of malodor, but as the capacity of these additives is finite, they require frequent reapplication (McCrory and Hobbs, 2001). Introduction of chemical feed additives to bind ammonia, to change the digest pH, to affect specific enzyme activities and to mask odor has either been costly or not consistently successful (Sutton et al., 1999).

Other possibilities for the reduction and control of malodor are biofiltration, ventilation systems and different systems for manure storage. Biofilters trap particles also provide an environment for biological degradation of the trapped compounds. They are effectively reducing odors but dust generated in facilities frequently leads to poor filter performance. Use of mechanical aerators on manure slurry would reduce odors substantively. However, during the process of oxygen incorporation nitrogen is volatized to the atmosphere, primarily as ammonia. Therefore, aeration although effective for reducing odor, can increase ammonia emissions (Iowa State University, http://www.extension.iastate.edu/Publications/PM1972a.pdf).

Recent research has therefore emphasized the manipulation of diet, (i) to increase nutrient utilization of the diet to reduce odorous excretion products, (ii) to enhance microbial metabolism in the intestinal tract thus reducing excretion of odor-causing compounds, and (iii) to change the physical characteristics of urine and feces to reduce odor emission (Sutton et al., 1993). Studies have shown that by reducing the protein levels in the diet and at the same time balancing with synthetic amino acids (Sutton et al, 1996, Hobbs et al. 1996, van Kempen, 2003, Portejoie et al. 2004) and by using high fiber diets, e.g. soybean hulls (Moeser et al., 2001) the amount of odorous compounds can be significantly reduced. In praxis, it is however not easy to reduce total protein in the diet since livestock is fed ad libitium to gain more product in a shorter period of time.

Gaseous emissions from slurries are affected by conditions such as temperature, oxygen content, humidity, air exchange rate, pH, buffering capacity and dry matter content of the slurry. pH is a very important modifier. Lowering the pH of urine and subsequent slurry is suggested to be beneficial for reducing odor and ammonia emissions. Maintaining the proper acid-base balance and buffering capacity of the diet and the intestinal contents may influence the final pH (Risley et al., 1992, van Kempen, 2001).

Thus, odor emission from animal facilities can be reduced through nutrition, but likely at the expense of higher feed costs which farmer are normally not willing to take.

Reduction of odorous substances by microorgansims has successfully used in biofilters. However, the aerobic microorganisms found in these filters are not suitable for application tin the anaerobic gastrointestinal tract since the degradation processes require oxygen. In addition, these microorganisms are not “generally regarded as safe” (GARS) organisms and thus difficult to be approved for animal use. Geng et al. (2004) isolated an aerobic bacterium from activated sludge that was able to degrade dimethylsulphide in vitro. Kim et al. (2004) isolated a phototrophic Rhodopseudomonas palustris strain that removed odorous organic acids when cultured in swine wastewater. Yet, phototrophic bacteria are anaerobic in light and thus not suitable for application in animal.

Yun and Otha (2005) immobilized an aerobic Rhodococcus strain that removed aqueous volatile fatty acids in wastewater. Yumoto et al. (2004) isolated a novel Bacillus strain from soil that deodorized short chain fatty acids. Naidu et al. (2002) selected a Lactobacillus casei strain that is able to reduce sulphide in vitro under growth conditions, but only low levels with a maximum of 341 ppm sulphide after 48 hours. In U.S. Pat. No. 4,345,032, and U.S. Pat. No. 4,879,238 Lactobacillus strains are disclosed which show a growth promotion in the presence of odorous substances like sulphides, ammonia or acetic acids.

To manipulate the existing microflora in situ, i.e. in the gastrointestinal tract of e.g. pigs, specific prebiotic substrates were introduced into the diet, or probiotic microbial cultures were administered to compete with the endogenous bacterial populations (Miner, 1995). Studies have shown that further addition of complex carbohydrates or organic acids to the diet can modulate the microflora in the digestive system of pigs (Sutton et al., 1991, Miner, 1995). For example, fructooligosaccharides have been shown to alter volatile fatty acid patterns in the gastrointestinal tract, reduce the total aerobes, increase the number of bifidobacteria (Houdijk et al., 2002) and reduce odorous compounds form swine manure (Hidaka et al., 1986). Miner (1975) summarized several studies showing, however, that attempts to reduce odors by feeding various microbial organisms were not successful. Ko et al. (2003) could show that feeding of a dietary probiotic for broilers increased emission of ammonia and hydrogen sulphide.

Thus, there is a need for means and methods allowing to effectively reduce the generation of feces odor, in particular, in the intestinal tract.

The present invention addresses this need and provides microorganisms, which reduce the generation of feces odor. In particular, it provides the embodiments as characterized in the claims.

Accordingly, the present invention in a first aspect relates to a microorganism which is able to reduce the generation of feces odor by decreasing the amount of at least one of the compounds selected from the group consisting of:

(i) a sulphide compound;
(ii) methyl mercaptan;
(iii) cadavarine;
(iv) putrescine;
(v) indole; and
(vi) skatole; and wherein said decrease in the amount of said compounds is independent of the growth of the microorganism.

The inventors for the first time identified microorganisms, which effectively reduce the amount of substances responsible for the generation of feces odor and provided methods for their identification. These microorganisms are able to decrease the amount or concentration of odorous compounds like, methyl mercaptan, sulphide compounds, cadaverine, putrescine, indole or skatole in the manure independently of their growth status, i.e. (i) the decrease in amount of these substances can take place even when the microorganism is not growing and (ii) these substances are not utilized as nutrients or energy sources. Thereby these microorganisms are able to reduce the generation of feces odor in a great variety of environments including those with variable supply of nutrients or environments, which do not contain nutrients.

The term “reducing the generation of feces odor” relates to the decrease in amount of odorous substances present in the feces. Preferably, the term relates to a decrease in amount of at least one of the compounds, methyl mercaptan a sulphide compound, cadaverine, putrescine, indole or skatole. The term “at least one of the compounds” means that one of the compounds (i) a sulphide compound, (ii) methyl mercaptan, (iii) cadaverine, (iv) putrescine, (iv) indole and (v) skatole alone is decreased in its amount or concentration in the feces. The term also means that any combination of compounds (i) to (v) is decreased in its amount or concentration in the feces by the microorganism of the invention. The term “combination” relates to a concomitant decrease in the amount or concentration of each grouping, permutation or sub-grouping of compounds (i) to (v) encompassed within the group of compounds as specified herein above. In a preferred embodiment, the term “combination” relates to a decrease in the amount or concentration of (i) a sulphide compound and (ii) methyl mercaptan, in another preferred embodiment the term relates to a decrease in the amount or concentration of (iii) cadaverine and (iv) putrescine, in yet another preferred embodiment the term relates to a decrease in the amount or concentration of (v) indole and (vi) skatole. In a more preferred embodiment, the term relates to a decrease in the amount or concentration of (i) a sulphide compound (iii) cadaverine and (iv) putrescine.

The term “feces” relates to waste materials, including bacteria, undigested food and sloughed-off intestinal cells or material produced from the intestines that are expelled from the intestinal tract through the anus. Preferably, the term relates to waste material or manure of animals. More preferably the term relates to waste material from companion animals, e.g. from cattle, horse, fowls, to waste material from domestic animals, e.g. from rabbits or guinea pigs or to waste material from human beings. Even more preferably, the term relates to waste material from dogs or cats. Most preferably, the term relates to waste material from pigs.

The term “feces odor” means that a typical manure odor can be detected. Preferably, the term means that the detection of the typical manure odor is verified by sniffing with the nose, preferably the nose of a skilled person.

The verification by “sniffing with the nose” relates to a detection of typical feces odor carried out by one or more persons having been trained for the detection of odor with their noses. The detection may be carried out in any suitable form or by using any suitable technique known to the person skilled in the art. Preferably the detection may be carried out by a qualified panel of persons having been trained for the detection of feces odor with their noses, more preferably it may be carried out by five persons or, most preferably, by eight persons which form a qualified panel. More preferably, a qualified panel for the detection of odor may consist of trained persons in accordance with the regulations provided in standard EN 13725 or VDI 3882. There are different categories of odor intensity. One possibility to define these categories is: 0=no odor detectable, 1=very faint odor detectable, 2=faint odor detectable, 3=distinct odor detectable and 4=strong odor detectable. Preferably, odor intensity may be measured in the following categories: 0=no odor detectable, 1=very faint odor detectable, 2=faint odor detectable, 3=distinct odor detectable and 4=strong odor detectable, 5=very strong odor detectable and 6=extremely strong odor detectable. More preferably, odor intensity may be measured in accordance with standard EN 13725 or VDI 3882. The person or persons forming the qualified panel may independently assess the odor intensity of odorous samples of feces. Preferably, the assessment is carried out in accordance with the regulations provided in standard EN 13725 or VDI 3882.

Preferably the odor of in vitro generated samples comprising the compounds methyl mercaptan, a sulphide compound, cadaverine, putrescine, indole or skatole or of ex vivo samples of feces and a microorganism able to reduce the generation of feces odor or corresponding control samples without microorganisms as defined in the invention may be assessed. The value of odor perception of the person(s) belonging to the qualified panel may be calculated by any means known to the person skilled in the art. Preferably, the mean value of odor perception of all person(s) belonging to the qualified panel may be calculated. Based on these data the intensity of odor may subsequently be quantified or evaluated by any means known to the person skilled in the art.

Another possibility to carry out odor quantification is the determination of an odor concentration in diluted odorous samples. The odor may be defined in such a quantification as odor units per volume, as known to the person skilled in the art, e.g. from Bunton et al., 2007, preferably in accordance with standard EN 13725 or VDI 3882. Preferably, one odor unit per m³ is an indication of the presence of odor, as perceived by a qualified panel of persons as described herein above, in a dilution of 1:1 of an odorous air sample vs. pure air. For instance, if the dilution 1:500 of an odorous air sample vs. pure air is recognized by a qualified panel of persons as described herein above, the odor is of a concentration of 500 odor units/m³ (OU/m³).

A further possibility to define odor is a system of hedonic tones by a qualified panel of persons as described herein above. The term “hedonic tone” means a property of an odor relating to its pleasantness or unpleasantness, as known to the person skilled in the art, e.g. from standard EN 13725 or VDI 3882. Preferably, an odor is evaluated by a qualified panel of persons as described herein above for its hedonic tone in the neutral context of, e.g., an olfactometric presentation and the panellist is exposed to a controlled stimulus in terms of intensity and duration. Preferably, the olfactometric presentation is carried out in accordance with the guidance provided in standard EN 13725 or VDI 3882. The degree of pleasantness or unpleasantness may be determined by each panellist's experience and emotional associations. There are different categories of odor character in the hedonic tone system. Preferably these categories may be defined as: +4=extremely pleasant, +3=very pleasant, +2=pleasant, +1=slightly pleasant, 0=neutral, −1=slightly unpleasant, −2=unpleasant, −3 very unpleasant and −4=extremely unpleasant.

The term “decrease in amount” relates to a decrease in the number of molecules of a sulphide compound, methyl mercaptan, cadaverine, putrescine, indole and/or skatole present in a mixture containing at least a sulphide compound, methyl mercaptan, cadaverine, putrescine, indole or skatole alone or any combination of these compounds and a microorganism according to the invention in comparison to a mixture in which the microorganism according to the invention is not present. The term “decrease” means that the amount of a sulphide compound, methyl mercaptan, cadaverine, putrescine, indole or skatole in a mixture containing at least a sulphide compound, methyl mercaptan, cadaverine, putrescine, indole and/or skatole and a microorganism according to the invention is 95%, 90%, 80%, 70%, 60%, 50%, 40%, 30%, 20%, 10%, 5%, 3% 2%, more preferably 1% and most preferably 0% of the amount of a sulphide compound, methyl mercaptan, cadaverine, putrescine, indole or skatole, respectively in a mixture in which the microorganism according to the invention is not present. The term “sulphide compound” relates to members of the family of sulphides consisting of, e.g., hydrogen sulphide, sodium sulphide, dimethyl sulphide, dimethyl disulphide, dimethyl trisulphide etc. Preferably, the term relates to hydrogen sulphide and sodium sulphide. Most preferably, the term relates to hydrogen sulphide.

The capability of a microorganism according to the invention to decrease the amount of a sulphide compound can be determined by methods known to the person skilled in the art. Said capability may be determined, for example, by an assay as described herein below, more preferably, as described in the Examples.

Briefly, such an assay comprises the following steps:
mixing a microorganism which should be tested for its capability decrease in amount of a sulphide compound with a medium or buffer containing a sulphide compound;
incubating the mixture under conditions allowing the decrease in amount of a sulphide compound;
extracting the supernatant; and
detecting the amount of a sulphide compound in the supernatant.

The mixing of the components may be carried out in any suitable proportion and in any suitable buffer or medium, known to the person skilled in the art. In a preferred embodiment a microorganism which is able to decrease the amount of a sulphide compound is anaerobically cultivated in MRS broth at 37° C. In a further preferred embodiment a microorganism which is able to decrease the amount of a sulphide compound is aerobically cultivated in YM broth at 30° C. The cultivation may be carried out, e.g., for 10 to 80 h, preferably for 15 to 60 h and more preferably for 24 to 48 h. In a most preferred embodiment the anaerobic cultivation may be carried out for 24 h. In a further most preferred embodiment the aerobic cultivation may be carried out for 48 h. As volume for the anaerobic or aerobic cultivation any volume suitable can be used, preferably a volume of 1 μl to 1 ml, more preferably 50 μl to 750 μl ml, even more preferably 100 to 300 μl, and most preferably 150 μl is used. The inoculation may be carried out by any means known to the person skilled in the art. Preferably, an inoculum of a freezing culture is used. More preferably, 1 to 100 μl of a freezing culture are used, most preferably 10 μl of a freezing culture are used.

The microorganism which is able to decrease the amount of a sulphide compound is subsequently separated from the culture medium by any suitable method, e.g. the culture of said microorganism can be centrifuged, for example at 4000 rpm for 15 min. As a further step the obtained microorganisms may be washed by any suitable means known to the person skilled in the art, preferably an obtained cell pellet is washed one to several times in a buffer, e.g. a PBS-buffer, pH 7.0. As a further step, the obtained cells may be resuspended in any suitable buffer, known to the person skilled in the art, preferably an obtained cell pellet is resuspended in, e.g. 150 μl of oxygen-poor PBS buffer, pH 7.0. Preferably, the PBS buffer is freshly boiled and cooled down on ice.

For the assay cells of the microorganism which is able to decrease the amount of a sulphide compound, preferably washed cells, are mixed with a sulphide compound, e.g. sodium sulphide, in any suitable proportion known to the person skilled in the art. In a preferred embodiment, 1 to 500 μl of washed cells are used, more preferably, 10 to 200 μl, even more preferably 30 to 100 μl and most preferably 50 μl are used. The sulphide compound may, e.g. be used in an end-concentration of 10 to 1000 μM, preferably of 50 to 500 μM, more preferably of 100 to 250 μM and most preferably of 200 μM. As a control any suitable buffer or medium instead of the cells, for instance, PBS-buffer or MRS medium in a suitable, corresponding amount may be added to the mixture as characterized herein above. The samples are incubated under conditions allowing the decrease of amount of a sulphide compound. Such conditions are known by the skilled person. More preferably, the samples are incubated at 37° C. under anaerobic conditions, for example, for 1 min to 5 h, even more preferably 10 min to 3 h, 20 min to 2 h and most preferably for 1 h. Afterwards the cells may be centrifuged.

The presence of a sulphide compound in the supernatant can be detected by methods known to the person skilled in the art. For example, the sulphide in the supernatant may be precipitated by any means known to the skilled artisan, e.g. with a zinc acetate solution. Preferably, 50 μl of a zinc acetate solution of a working solution of 1 part stock solution and 5 parts aqua dest., freshly boiled and cooled down on ice with a stock solution of 182 mM zinc acetate in 2% acetic acid are used. As a further step, a DMPD/ferric chloride solution, for example a, working solution of 1 part stock solution+9 parts 6 M HCl with a stock solution of 180 mM DMPD (N,N-Dimethyl-1,4-phenylenediamine sulphate, Sigma), 540 mM FeCl₃, solved in 6 M HCl; is added.

The solution may then be incubated under conditions known to the person skilled in the art, e.g. for 30 min at room temperature under light protection, yielding a methylene blue staining.

The presence of a sulphide compound can be detected by methods known to the person skilled in the art. Preferably, it is detected by a photometrical measurement of methylene blue, e.g. at a wavelength of 678 nm. The absorption can be used as a measurement of the amount or concentration of a sulphide compound. A microorganism is regarded as being able to decrease the amount of a sulphide compound if the amount of a sulphide compound in such a sulphide reduction assay with at least one such microorganism is not more than 95%, 90%, 80%, 70%, 60%, 50%, 40%, 30%, 20%, 10%, 5%, 3%, 2%, preferably not more than 1% and most preferably not more than 0% of the amount of a sulphide compound that is detectable in a mixture in which the microorganism according to the invention is not present.

The described assay may also be used to identify microorganisms, which are capable of decreasing the amount of a sulphide compound.

The capability of a microorganism according to the invention to decrease the amount of methyl mercaptan can be determined according to methods well known to the person skilled in the art. Said capability may be determined, for example, by an assay as described herein below, more preferably, as described in the Examples.

Briefly, such an assay comprises the following steps:
mixing a microorganism which should be tested for its capability decrease the amount of methyl mercaptan with a medium or buffer containing methyl mercaptan;
incubating the mixture under conditions allowing the decrease in amount of methyl mercaptan;
extracting the supernatant; and
detecting the amount of methyl mercaptan in the supernatant.

The mixing of the components may be carried out in any suitable proportion and in any suitable buffer or medium, known to the person skilled in the art. In a preferred embodiment a microorganism which is able to decrease the amount of methyl mercaptan is anaerobically cultivated in MRS broth at 37° C. In a further preferred embodiment a microorganism which is able to decrease the amount of methyl mercaptan is aerobically cultivated in YM broth at 30° C. The cultivation may be carried out, e.g., for 10 to 80 h, preferably for 15 to 60 h and more preferably for 24 to 48 h. In a most preferred embodiment the anaerobic cultivation may be carried out for 24 h. In a further most preferred embodiment the aerobic cultivation may be carried out for 48 h. As volume for the anaerobic or aerobic cultivation any volume suitable can be used, preferably a volume of 1 μl to 1 ml, more preferably 50 μl to 750 μl ml, even more preferably 100 to 300 μl, and most preferably 150 μl is used. The inoculation may be carried out by any mean known to the person skilled in the art. Preferably, an inoculum of a freezing culture is used. More preferably, 1 to 100 μl of a freezing culture are used, most preferably 10 μl of a freezing culture are used.

The microorganism which is able to decrease the amount of methyl mercaptan is subsequently separated from the culture medium by any suitable method, e.g. the culture of said microorganism can be centrifuged, for example at 4000 rpm for 15 min. As a further step the obtained microorganisms may be washed by any suitable means known to the person skilled in the art, preferably an obtained cell pellet is washed one to several times in a buffer, e.g. a PBS-buffer, pH 7.0. As a further step, the obtained cells may be resuspended in any suitable buffer, known to the person skilled in the art, preferably an obtained cell pellet is resuspended in, e.g. 150 μl of phosphate buffer, pH 8.0. Preferably, the PBS buffer is 50 mM sodium phosphate at pH 8.0.

For the assay cells of the microorganism, which is able to decrease the amount of methyl mercaptan, preferably washed cells, are mixed with methyl mercaptan in any suitable proportion known to the person skilled in the art. In a preferred embodiment, 1 to 500 μl of washed cells are used, more preferably, 10 to 200 μl, even more preferably 30 to 100 μl and most preferably 50 μl are used. The methyl mercaptan may, e.g. be used in an end-concentration of 10 to 2000 μM, preferably of 50 to 1000 μM, more preferably of 100 to 750 μM and most preferably of 500 μM. The methyl mercaptan may, for example, be dissolved in a phosphate/DMSO solution, preferably in a phosphate buffer and 10% DMSO. As a control any suitable buffer or medium instead of the cells, for instance, phosphate buffer in a suitable, corresponding amount may be added to the mixture as characterized herein above. The samples are incubated under conditions allowing the decrease of amount of methyl mercaptan. Such conditions are known by the skilled person. Preferably, the samples are incubated at 37° C. under anaerobic conditions, for example, for 1 min to 5 h, even more preferably 10 min to 3 h, 20 min to 2 h and most preferably for 1 h. Afterwards the cells may be centrifuged.

The presence of methyl mercaptan in the supernatant can be detected by methods known to the person skilled in the art. For example, the supernatant may be derivatised with any means known to the skilled artisan, e.g. with a DTNB solution. Preferably, 180 μl of a DTNB solution of a working solution of 1 part stock solution+19 parts phosphate with a stock solution of 5 mM DTNB (5,5″-Dithiobis(2-nitrobenzoic acid), Sigma) in phosphate buffer are used.

The solution may then be incubated under conditions known to the person skilled in the art, e.g. for 30 min at room temperature under light protection, yielding a yellow reduction product staining.

The presence of methyl mercaptan can be detected by methods known to the person skilled in the art. Preferably, it is detected by a photometrical measurement of the yellow reduction product, e.g. at a wavelength of 405 nm. The absorption can be used as a measurement of the amount or concentration of methyl mercaptan. A microorganism is regarded as being able to decrease the amount of methyl mercaptan if the amount of methyl mercaptan in such a methyl mercaptan reduction assay with at least one such microorganism is not more than 95%, 90%, 80%, 70%, 60%, 50%, 40%, 30%, 20%, 10%, 5%, 3%, 2%, preferably not more than 1% and most preferably not more than 0% of the amount of methyl mercaptan that is detectable in a mixture in which the microorganism according to the invention is not present.

The described assay may also be used to identify microorganisms, which are capable of decreasing the amount of methyl mercaptan.

The capability of a microorganism according to the invention to decrease the amount of cadaverine or putrescine can be determined according to methods well known to the person skilled in the art. Said capability may be determined, for example, by an “Biogen amine reduction assay” as described herein below, more preferably, as described in the Examples.

Briefly, such an assay comprises the following steps:
mixing a microorganism which should be tested for its capability to decrease the amount of cadaverine or putrescine with a medium or buffer containing cadaverine and/or putrescine;
incubating the mixture under conditions allowing the decrease in amount of cadaverine or putrescine;
extracting the supernatant; and
detecting the amount of cadaverine or putrescine in the supernatant.

The mixing of the components may be carried out in any suitable proportion and in any suitable buffer or medium, known to the person skilled in the art. In a preferred embodiment a microorganism which is able to decrease the amount of cadaverine or putrescine is anaerobically cultivated in MRS broth at 37° C. In a further preferred embodiment a microorganism which is able to decrease the amount of cadaverine or putrescine is aerobically cultivated in YM broth at 30° C. The cultivation may be carried out, e.g., for 10 to 80 h, preferably for 15 to 60 h and more preferably for 24 to 48 h. In a most preferred embodiment the anaerobic cultivation may be carried out for 24 h. In a further most preferred embodiment the aerobic cultivation may be carried out for 48 h. As volume for the anaerobic or aerobic cultivation any volume suitable can be used, preferably a volume of 1 μl to 1 ml, more preferably 50 μl to 750 μl ml, even more preferably 100 to 300 μl, and most preferably 150 μl is used. The inoculation may be carried out by any mean known to the person skilled in the art. Preferably, an inoculum of a freezing culture is used. More preferably, 1 to 100 μl of a freezing culture are used, most preferably 10 μl of a freezing culture are used.

The microorganism which is able to decrease the amount of cadaverine or putrescine is subsequently separated from the culture medium by any suitable method, e.g. the culture of said microorganism can be centrifuged, for example at 4000 rpm for 15 min. As a further step the obtained microorganisms may be washed by any suitable means known to the person skilled in the art, preferably an obtained cell pellet is washed one to several times in a buffer, e.g. a PBS-buffer, pH 7.0. As a further step, the obtained cells may be resuspended in any suitable buffer, known to the person skilled in the art, preferably an obtained cell pellet is resuspended in, e.g. 150 μl of a PBS buffer (10 mM phosphate, 150 mM NaCl, pH 7.0).

For the assay cells of the microorganism, which is able to decrease the amount of cadaverine or putrescine, preferably washed cells, are mixed with cadaverine and/or putrescine in any suitable proportion known to the person skilled in the art. In a preferred embodiment, 1 to 500 μl of washed cells are used, more preferably, 10 to 200 μl, even more preferably 30 to 100 μl and most preferably 50 μl are used. The cadaverine or putrescine may, e.g., be used in an end-concentration of 1 to 1000 μM, preferably of 10 to 500 μM, more preferably of 20 to 100 μM and most preferably of 50 μM in the sample. In a preferred embodiment cadaverine and putrescine may be present in the sample at the same time. The cadaverine or putrescine may, for example, be dissolved in any buffer known to the person skilled in the art, preferably in a PBS buffer. As a control any suitable buffer or medium instead of the cells, for instance, phosphate buffer in a suitable, corresponding amount may be added to the mixture as characterized herein above. The samples are incubated under conditions allowing the decrease of amount of cadaverine or putrescine. Such conditions are known by the skilled person. Preferably, the samples are incubated at 37° C. under anaerobic conditions, for example, for 1 min to 5 h, even more preferably 10 min to 3 h, 20 min to 2 h and most preferably for 1 h. More preferably, the cells may be shaken during the incubation, e.g. at 140 rpm. Afterwards the cells may be centrifuged.

The presence of cadaverine and/or putrescine in the supernatant can be detected by methods known to the person skilled in the art. For example, the supernatant may be derivatised with any means known to the skilled artisan, e.g. with a NBD-chloride solution using propyl amine as an internal standard. Preferably, 40 μl of a freshly prepared NBD-chloride solution (e.g. at a concentration of 2 mg NBD-Cl/ml ethanol) and 80 μl propyl amine solution (e.g. at a concentration of 50 μM propyl amine in tetra-borate buffer at pH 9.75) are used.

The solution may then be incubated under conditions known to the person skilled in the art, e.g. for 60 min at a temperature of 60° C., afterwards cooled down to room temperature, for example in an ice bath. Subsequently, the pH of the sample may be adjusted by any means known to the skilled artisan, e.g. to pH 6-pH 7.

The presence of cadaverine and/or putrescine can be detected by methods known to the person skilled in the art. Preferably, it is detected by a HPLC/FL analysis. More preferably, the quantity of cadaverine and/or putrescine is observed by HPLC analysis performed on an Agilent chemstation with any column known to the person skilled in the art, e.g. a Supelco Ascentis RP-AMIDE column (15 cm×3 mm, 5 μm). As solvent gradient every solvent gradient suitable, as known to the person skilled in the art, may be used. Preferably, a solvent gradient of: 0 min: 15% acetonitrile/85% citrate buffer pH 3.0, 3 min: 20% acetonitrile/80% citrate buffer pH 3.0, 11 min: 85% acetonitrile/15% citrate buffer pH 3.0, 12 min: 85% acetonitrile/15% citrate buffer pH 3.0, 16 min: 15% acetonitrile/85% citrate buffer pH 3.0, with a stop after 17 min may be used. The column temperature may be any temperature known to be suitable to the skilled person, e.g. 20° C. The constant flow velocity may be at any suitable value known to the person skilled in the art, for example at 1.2 ml/min. The presence of cadaverine and/or putrescine can be detected by methods known to the person skilled in the art. Preferably, it is detected by fluorescence analysis (λₘₐₓ=490 nm, λₑₘ=550 nm) and comparison of retention time to the pure standard substances. The peak area may be used as a measure for the concentration of cadaverine or putrescine.

A microorganism is regarded as being able to decrease the amount of cadaverine or putrescine if the amount of cadaverine or putrescine in such a biogen amine reduction assay with at least one such microorganism is not more than 95%, 90%, 80%, 70%, 60%, 50%, 40%, 30%, 20%, 10%, 5%, 3%, 2%, preferably not more than 1% and most preferably not more than 0% of the amount of cadaverine or putrescine that is detectable in a mixture in which the microorganism according to the invention is not present.

The capability of a microorganism according to the invention to decrease the amount of indole or skatole can be determined according to methods well-known to the person skilled in the art. Said capability may be determined, for example, by an “Indole reduction assay” as described herein below, more preferably, as described in the Examples.

Briefly, such an assay comprises the following steps:
mixing a microorganism which should be tested for its capability to decrease the amount of indole or skatole with a medium or buffer containing indole and/or skatole;
incubating the mixture under conditions allowing the decrease in amount of indole or skatole;
extracting the supernatant; and
detecting the amount of indole or skatole in the supernatant.

The mixing of the components may be carried out in any suitable proportion and in any suitable buffer or medium, known to the person skilled in the art. In a preferred embodiment a microorganism, which is able to decrease the amount of indole or skatole is anaerobically cultivated in MRS broth at 37° C. In a further preferred embodiment a microorganism, which is able to decrease the amount of indole or skatole is aerobically cultivated in YM broth (Difco Manual; 3.0 g yeast extract, 3.0 g malt extract, 5.0 g peptone, 10.0 g dextrose per liter) at 30° C. The cultivation may be carried out, e.g., for 10 to 80 h, preferably for 15 to 60 h and more preferably for 24 to 48 h. In a most preferred embodiment the anaerobic cultivation may be carried out for 24 h. In a further most preferred embodiment the aerobic cultivation may be carried out for 48 h. As volume for the anaerobic or aerobic cultivation any volume suitable can be used, preferably a volume of 1 μl to 1 ml, more preferably 50 μl to 750 μl ml, even more preferably 100 to 300 μl, and most preferably 150 μl is used. The inoculation may be carried out by any means known to the person skilled in the art. Preferably, an inoculum of a freezing culture is used. More preferably, 1 to 100 μl of a freezing culture are used, most preferably 10 μl of a freezing culture are used.

The microorganism which is able to decrease the amount of indole or skatole is subsequently separated from the culture medium by any suitable method, e.g. the culture of said microorganism can be centrifuged, for example at 4000 rpm for 15 min. As a further step the obtained microorganisms may be washed by any suitable means known to the person skilled in the art, preferably an obtained cell pellet is washed one to several times in a buffer, e.g. a PBS-buffer, pH 7.0. As a further step, the obtained cells may be resuspended in any suitable buffer, known to the person skilled in the art, preferably an obtained cell pellet is resuspended in, e.g. 150 μl of phosphate buffer, preferably a PBS buffer.

For the assay cells of the microorganism, which is able to decrease the amount of indole or skatole, preferably washed cells, are mixed with indole and/or skatole in any suitable proportion known to the person skilled in the art. In a preferred embodiment, 1 to 500 μl of washed cells are used, more preferably, 10 to 200 μl, even more preferably 30 to 100 μl and most preferably 50 μl are used. The indole or skatole may, e.g. be used in an end-concentration of 1 to 1000 μM, preferably of 10 to 500 μM, more preferably of 20 to 400 μM and most preferably of 200 μM in the sample. In a preferred embodiment indole and skatole may be present in the sample at the same time. The indole or skatole may, for example, be dissolved in any buffer known to the person skilled in the art, preferably in a PBS buffer. As a control any suitable buffer or medium instead of the cells, for instance, phosphate buffer in a suitable, corresponding amount may be added to the mixture as characterized herein above. The samples are incubated under conditions allowing the decrease of amount of indole or skatole. Such conditions are known by the skilled person. Preferably, the samples are incubated at 37° C. under anaerobic conditions, for example, for 1 h to 30 h, even more preferably 2 h to 24 h, 3 h to 20 h and most preferably for 16 h. More preferably, the cells may be shaken during the incubation, e.g. at 140 rpm. Afterwards the cells may be centrifuged.

The presence of indole and/or skatole in the supernatant can be detected by methods known to the person skilled in the art. Preferably, it is detected by a HPLC/DAD analysis. More preferably, the quantity of indole and/or skatole is observed by HPLC analysis performed on an Agilent chemstation with any column known to the person skilled in the art, e.g. an Agilent Zorbax Eclipse XDB-C8 column (15 cm×3 mm, 5 μm). As isocratic program any isocratic program suitable, as known to the person skilled in the art, may be used. Preferably, an isocratic program of: 40% 0.1 M sodium acetate/45% acetonitrile/15% methanol pH 7.2 for 4 min may be used. The column temperature may any temperature suitable, as known to the skilled person, e.g. 25° C. The constant flow velocity may be at any suitable value, known to the person skilled in the art, for example at 1 ml/min. The presence of indole and/or skatole can be detected by methods known to the person skilled in the art. Preferably, it is detected by DAD analysis (λ=220 nm) and comparison of retention time to the pure standard substances. The peak area may be used as a measure for the concentration of indole or skatole.

A microorganism is regarded as being able to decrease the amount of indole or skatole if the amount of indole or skatole in such a indole reduction assay with at least one such microorganism is not more than 95%, 90%, 80%, 70%, 60%, 50%, 40%, 30%, 20%, 10%, 5%, 3%, 2%, preferably not more than 1% and most preferably not more than 0% of the amount of indole or skatole that is detectable in a mixture in which the microorganism according to the invention is not present.

The capability of a microorganism according to the invention to decrease the amount of odorous substances present in the feces may also be determined in ex vivo feces according to methods well-known to the person skilled in the art. The term “ex vivo” means that the feces have been obtained from living animals, preferably from companion animals like cattle, horse, fowls, from domestic animals like cats, rabbits or guinea pigs or from human beings. More preferably, the term “living animal” refers to pigs or dogs. Said capability may be determined, for example, by an “Feces odor reduction assay” as described herein below, more preferably, as described in the Examples.

Briefly, such an assay comprises the following steps:
mixing a microorganism which should be tested for its capability to decrease the amount of odorous substances present in the feces with ex vivo feces;
incubating the mixture under conditions allowing the decrease in amount of odorous substances present in the feces;
extracting air samples; and
detecting the amount of odor in the sample by a sniffing test of a qualified panel as described herein above.

The mixing of the components may be carried out in any suitable proportion and in any suitable buffer or medium, known to the person skilled in the art. In a preferred embodiment a microorganism which is able to decrease the amount of odorous substances present in the feces is anaerobically cultivated in MRS broth at 37° C. In a further preferred embodiment a microorganism which is able to decrease the amount of odorous substances present in the feces is aerobically cultivated in YM broth at 30° C. The cultivation may be carried out, e.g., for 5 to 80 h, preferably for 10 to 60 h and more preferably for 12 to 48 h. In a most preferred embodiment the anaerobic cultivation may be carried out for 16 h. In a further most preferred embodiment the aerobic cultivation may be carried out for 24 h. As volume for the anaerobic or aerobic cultivation any volume suitable can be used, preferably a volume of 1 μl to 5 ml, more preferably 50 μl to 3 ml, even more preferably 100 to 2 ml, and most preferably 1 ml is used. The inoculation may be carried out by any means known to the person skilled in the art. Preferably, an inoculum of a freezing culture is used. More preferably, 1 to 100 μl of a freezing culture are used, most preferably 10 μl of a freezing culture are used.

The microorganism which is able to decrease the amount of odorous substances present in the feces is subsequently separated from the culture medium by any suitable method, e.g. the culture of said microorganism can be centrifuged, for example at 4000 rpm for 15 min. As a further step the obtained microorganisms may be washed by any suitable means known to the person skilled in the art, preferably an obtained cell pellet is washed one to several times in a buffer, e.g. a PBS-buffer, pH 7.0. As a further step, the obtained cells may be resuspended in any suitable buffer, known to the person skilled in the art, preferably an obtained cell pellet is resuspended in, e.g. 1 ml of phosphate buffer, preferably a PBS buffer.

As feces any animal feces known to the skilled person may be used, preferably feces from companion animals like cattle, horse, fowls, from domestic animals like rabbits or guinea pigs or from human beings is used. More preferably, feces from dogs or cats is used. Most preferably, feces from pigs is used. The feces is obtained directly from the animal. Typically, the animals are fed according to any suitable diet, as known to the person skilled in the art. For instance, an animal diet comprises the following components: starch or energy sources (for example: corn, wheat or barley (rye)), protein sources (for example: soybean meal, rapseed meal, sunflower seed meal), DL methionine, L lysine HCl, limestone, mono-calcium-phosphate, salt, choline chloride (50%), vitamin premix, trace element premix, TiO₂. Preferably, an animal diet comprises the following ingredients: starch or energy sources (corn, wheat, barley or rye in a concentration of 644 g/kg feed), protein sources (soybean meal, rapseed meal or sunflower seed meal in a concentration of 300 g/kg feed), DL methionine in a concentration of 0.3 g/kg feed, L lysine HCl in a concentration of 2.5 g/kg feed, limestone in a concentration of 0.4 g/kg feed, mono-calcium-phosphate in a concentration of 3.8 g/kg feed, salt in a concentration of 12.7 g/kg feed, choline chloride (50%) in a concentration of 1 g/kg feed, vitamin premix in a concentration of 4 g/kg feed, trace element premix in a concentration of 0.8 g/kg feed, TiO₂ in a concentration of 1 g/kg feed. More preferably, the herein above described animal diet is a diet for pigs.

For the assay cells of the microorganism, which is able to decrease the amount of odorous substances present in the feces, preferably washed cells, are mixed with feces in any suitable proportion known to the person skilled in the art. In a preferred embodiment, 10⁵ to 10¹¹ of washed cells are used, more preferably, 10⁶ to 10¹⁰, even more preferably 10⁷ to 10⁹ and most preferably 10⁸ of washed cells are used. Feces are used in any suitable amount known to the person skilled in the art. Preferably an amount of 10 g to 100 g feces may be used, more preferably 25 g to 75 g feces may be used, most preferably 50 g feces may be used. The feces may be in any suitable condition known to the skilled person, preferably fresh feces is used. “Fresh feces” means that the feces is no older than 8 h, more preferably no older than 4 h, even more preferably no older than 2 h and most preferably no older than 1 h. For the assay the cells of the microorganism and the feces are mixed in any suitable medium known to the skilled person, preferably in water. The mixture is carried out in any suitable volume, known to the skilled person, preferrably a volume of 1 liter is used. In a preferred embodiment 10⁸ cells are mixed with 50 g feces in 1 liter of water.

As a control any suitable buffer or medium instead of the cells, for instance, phosphate buffer in a suitable, corresponding amount may be added to the mixture as characterized herein above. The samples are incubated under conditions allowing the decrease of amount of odorous substances present in the feces. Such conditions are known to the skilled person. Preferably, the samples are incubated at 37° C. under aerobic conditions, for example, for 0.5 h to 6 h, even more preferably for 1 h to 5 h, 2 h to 4 h and most preferably for 3 h. The incubation is carried out in any airtight container known to the person skilled in the art. The incubation may be carried out in any suitable manner known to the skilled person, preferably without agitation. The container may have any suitable volume known to the skilled person, preferably the container has a volume of 25 liters. The container may be filled or refilled with any suitable medium known to the skilled person, preferably, the container is filled or refilled with pure or odorless air.

Subsequently, the air is extracted from the container by any suitable means known to the skilled person and transferred by any suitable means known to the skilled person to a further container, preferably an airtight and inert bag, for instance a Nalophan bag.

The presence of odorous substances in the sample can be detected by any methods known to the person skilled in the art. Preferably, it is detected by an odor concentration assay, as known to the skilled person. More preferably, the assessment is carried out in accordance with the regulations provided in standard EN 13725 or VDI 3882. Air samples may be diluted in pure air via different dilution steps to different concentrations of odorous substances by any suitable means known to the skilled person, preferably by an olfactometer. A qualified panel as described herein above may subsequently test the diluted samples and indicate at which dilution an odor is still perceivable. The odor concentration may be measured in any suitable units, preferably in odor units per m³ (OU/m³) as described herein above. For instance, if the dilution of 1:500 of the odor sample vs. pure air is recognized as odor by the panellist, the odor concentration of the sample may be defined to be 500 odor units/m³ (OU/m³). For instance, if the dilution of 1:1000 of the odor sample vs. pure air is recognized as odor by the panellist, the odor concentration of the sample may be defined to be 1000 odor units/m³ (OU/m³).

In a further preferred embodiment the presence of odorous substances in the sample may be detected by a hedonic assay, as known to the skilled person. Preferably, the assessment is carried out in accordance with the regulations provided in standard EN 13725 or VDI 3882. Air samples may be diluted in pure air via different dilution steps to different concentrations of odorous substances by any suitable means known to the skilled person, preferably by an olfactometer. A qualified panel as described herein above may subsequently test the diluted samples and assign marks with respect to the pleasantness or hedonic tone as described herein above of the odor at the different dilutions. The degree of pleasantness or unpleasantness may be determined by each panellist's experience and emotional associations according to different categories of odor character in the hedonic tone system, which are: +4=extremely pleasant, +3=very pleasant, +2=pleasant, +1=slightly pleasant, 0=neutral, −1=slightly unpleasant, −2=unpleasant, −3 very unpleasant and −4=extremely unpleasant.

A microorganism is regarded as being able to decrease the amount of odorous substances present in the feces if the detectable odor in such an odor concentration or hedonic tone assay with at least one such microorganism is not more than 95%, 90%, 80%, 70%, 60%, 50%, 40%, 30%, 20%, 10%, 5%, 3%, 2%, preferably not more than 1% and most preferably not more than 0% of the odor that is detectable in a mixture in which the microorganism according to the invention is not present. Preferably, a microorganism is regarded as being able to decrease the amount of odorous substances present in the feces if the detectable odor in such a hedonic tone assay with at least one such microorganism is at least 0.25 points, preferably 0.5 points, more preferably 0.75 points, even more preferably 1.0 points and most preferably 2.0 points higher according to the hedonic tone system as described herein above in comparison to the odor that is detectable in a mixture in which the microorganism according to the invention is not present.

The capability of a microorganism according to the invention to decrease the amount of odorous substances present in the feces over an extended period of time may be determined in ex vivo feces according to methods well-known to the person skilled in the art. Said capability may be determined, for example, by an “Feces odor reduction assay over an extended period of time” as described herein below, more preferably, as described in the Examples.

Briefly, such an assay comprises the following steps:
mixing a microorganism which should be tested for its capability to decrease the amount of odorous substances present in the feces with ex vivo feces;
incubating the mixture under conditions over an extended period of time allowing the decrease in amount of odorous substances present in the feces;
extracting air samples at different time intervals; and
detecting the amount of odor in the sample by a sniffing test of a qualified panel as described herein above.

The mixing of the components may be carried out in any suitable proportion and in any suitable buffer or medium, known to the person skilled in the art. In a preferred embodiment a microorganism which is able to decrease the amount of odorous substances present in the feces is anaerobically cultivated in MRS broth at 37° C. In a further preferred embodiment a microorganism which is able to decrease the amount of odorous substances present in the feces is aerobically cultivated in YM broth at 30° C. The cultivation may be carried out, e.g., for 5 to 80 h, preferably for 10 to 60 h and more preferably for 12 to 48 h. In a most preferred embodiment the anaerobic cultivation may be carried out for 16 h. In a further most preferred embodiment the aerobic cultivation may be carried out for 24 h. As volume for the anaerobic or aerobic cultivation any volume suitable can be used, preferably a volume of 1 μl to 5 ml, more preferably 50 μl to 3 ml, even more preferably 100 to 2 ml, and most preferably 1 ml is used. The inoculation may be carried out by any means known to the person skilled in the art. Preferably, an inoculum of a freezing culture is used. More preferably, 1 to 100 μl of a freezing culture are used, most preferably 10 μl of a freezing culture are used.

The microorganism which is able to decrease the amount of odorous substances present in the feces is subsequently separated from the culture medium by any suitable method, e.g. the culture of said microorganism can be centrifuged, for example at 4000 rpm for 15 min. As a further step the obtained microorganisms may be washed by any suitable means known to the person skilled in the art, preferably an obtained cell pellet is washed one to several times in a buffer, e.g. a PBS-buffer, pH 7.0. As a further step, the obtained cells may be resuspended in any suitable buffer, known to the person skilled in the art, preferably an obtained cell pellet is resuspended in, e.g. 1 ml of phosphate buffer, preferably a PBS buffer.

As feces any animal feces known to the skilled person may be used, preferably feces from companion animals like cattle, horse, fowls, from domestic animals like rabbits or guinea pigs or from human beings is used. More preferably, feces from dogs or cats is used. Most preferably, feces from pigs is used. The feces is obtained directly from the animal. Typically, the animals are fed according to any suitable diet, as known to the person skilled in the art. For instance, an animal diet comprises the following components: starch or energy sources (for example: corn, wheat or barley (rye)), protein sources (for example: soybean meal, rapseed meal, sunflower seed meal), DL methionine, L lysine HCl, limestone, mono-calcium-phosphate, salt, choline chloride (50%), vitamin premix, trace element premix, TiO₂. Preferably, an animal diet comprises the following ingredients: starch or energy sources (corn, wheat or barley (rye) in a concentration of 644 g/kg), protein sources (soybean meal, rapseed meal or sunflower seed meal in a concentration of 300 g/kg), DL methionine in a concentration of 0.3 g/kg, L lysine HCl in a concentration of 2.5 g/kg, limestone in a concentration of 0.4 g/kg, mono-calcium-phosphate in a concentration of 3.8 g/kg, salt in a concentration of 12.7 g/kg, choline chloride (50%) in a concentration of 1 g/kg, vitamin premix in a concentration of 4 g/kg, trace element premix in a concentration of 0.8 g/kg, TiO₂ in a concentration of 1 g/kg. More preferably, the herein above described animal diet is a diet for pigs.

For the assay cells of the microorganism, which is able to decrease the amount of odorous substances present in the feces, preferably washed cells, are mixed with feces in any suitable proportion known to the person skilled in the art. In a preferred embodiment, 10⁵ to 10¹¹ of washed cells are used, more preferably, 10⁶ to 10¹⁰, even more preferably 10⁷ to 10⁹ and most preferably 10⁸ of washed cells are used. Feces are used in any suitable amount known to the person skilled in the art. Preferably an amount of 10 to 100 g feces may be used, more preferably 25 to 75 g feces may be used, most preferably 50 g feces may be used. The feces may be in any suitable condition known to the skilled person, preferably fresh feces is used. “Fresh feces” means that the feces is no older than 8 h, more preferably no older than 4 h, even more preferably no older than 2 h and most preferably no older than 1 h. For the assay the cells of the microorganism and the feces are mixed in any suitable medium known to the skilled person, preferably in water. The mixture is carried out in any suitable volume, known to the skilled person, preferably a volume of 1 liter is used. In a preferred embodiment 10⁸ cells are mixed with 50 g feces in 1 liter of water.

As a control any suitable buffer or medium instead of the cells, for instance, phosphate buffer in a suitable, corresponding amount may be added to the mixture as characterized herein above. The samples are incubated under conditions allowing the decrease of amount of odorous substances present in the feces. Such conditions are known to the skilled person. Preferably, the samples are incubated at 37° C. under aerobic conditions, for an extended period of time, for example 12 h to 96 h, more preferably 15 h to 48 h, even more preferably 20 h to 30 h and most preferably for 24 h. The incubation may be carried out in any airtight container known to the person skilled in the art. The incubation may be carried out in any suitable manner known to the skilled person, preferably without agitation. The container may have any suitable volume known to the skilled person, preferably the container has a volume of 25 liters. The container may be filled or refilled with any suitable medium known to the skilled person, preferably, the container is filled or refilled with pure or odorless air.

Subsequently, the air is extracted at certain time intervals from the container by any suitable means known to the skilled person and transfered by any suitable means known to the skilled person to a further container, preferably an airtight and inert bag, for instance a Nalophan bag. The time intervals may be, for example, every 1, 2, 3, 5, 6, 8, 10 or 24 h. In a preferred embodiment air samples are taken after 3 h, 6 h and 24 h. After extracting air from the container, the container is refilled with odorless air.

The presence of odorous substances in the sample can be detected by any methods known to the person skilled in the art. Preferably, it is detected by an odor concentration assay, as known to the skilled person. More preferably, the assessment is carried out in accordance with the regulations provided in standard EN 13725 or VDI 3882. Air samples may be diluted in pure air via different dilution steps to different concentrations of odorous substances by any suitable means known to the skilled person, preferably by an olfactometer. A qualified panel as described herein above may subsequently test the diluted samples and indicate at which dilution an odor is still perceivable. The odor concentration may be measured in any suitable units, preferably in odor units per m³ (OU/m³) as described herein above. For instance, if the dilution of 1:500 of the odor sample vs. pure air is recognized as odor by the panellist, the odor concentration of the sample may be defined to be 500 odor units/m³ (OU/m³). For instance, if the dilution of 1:1000 of the odor sample vs. pure air is recognized as odor by the panellist, the odor concentration of the sample may be defined to be 1000 odor units/m³ (OU/m³).

In a further preferred embodiment the presence of odorous substances in the sample may be detected by a hedonic assay, as known to the skilled person. Preferably, the assessment is carried out in accordance with the regulations provided in standard EN 13725 or VDI 3882. Air samples may be diluted in pure air via different dilution steps to different concentrations of odorous substances by any suitable means known to the skilled person, preferably by an olfactometer. A qualified panel as described herein above may subsequently test the diluted samples and assign marks with respect to the pleasantness or hedonic tone as described herein above of the odor at the different dilutions. The degree of pleasantness or unpleasantness may be determined by each panellist's experience and emotional associations according to different categories of odor intensity in the hedonic tone system, which are: +4=extremely pleasant, +3=very pleasant, +2=pleasant, +1=slightly pleasant, 0=neutral, −1=slightly unpleasant, −2=unpleasant, −3 very unpleasant and −4=extremely unpleasant.

A microorganism is regarded as being able to decrease the amount of odorous substances present in the feces if the detectable odor in such an odor concentration or hedonic tone assay with at least one such microorganism is not more than 95%, 90%, 80%, 70%, 60%, 50%, 40%, 30%, 20%, 10%, 5%, 3%, 2%, preferably not more than 1% and most preferably not more than 0% of the odor that is detectable in a mixture in which the microorganism according to the invention is not present. Preferably, a microorganism is regarded as being able to decrease the amount of odorous substances present in the feces if the detectable odor in such a hedonic tone assay with at least one such microorganism is at least 0.25 points, preferably 0.5 points, more preferably 0.75 points, even more preferably 1.0 points and most preferably 2.0 points higher according to the hedonic tone system as described herein above in comparison to the odor that is detectable in a mixture in which the microorganism according to the invention is not present.

The term “independent of the growth of the microorganism” means that the decrease of at least one of the compounds selected from the group consisting of (i) a sulphide compound, (ii) methyl mercaptan, (iii) cadaverine, (iv) putrescine, (v) indole, and (vi) skatole occurs without a concomitant growth promotion of the microorganism due to the reduction of said compounds. Thus, the microorganisms of the present invention do not show any growth if they are cultivated under conditions which do not support growth of the microorganisms and if they are simultaneously provided with at least one of the above-mentioned compounds although the microorganisms lead to a decrease of the amount of at least one of the above-mentioned compounds. The term “growth” or “growing” means an increase in biomass, cell size and/or cell number per time unit. The growth of a microorganism can be determined by any means known to a person skilled in the art, for example cell counting or the measurement of optical density.

Preferably the growth of absence of growth of a microorganism may be determined in an assay in which a microorganism is incubated under specific conditions known to a person skilled in the art in a specific medium or buffer known to a person skilled in the art. The growth or absence of growth can thus be determined by, e.g., photometrically measuring the optical density of the microorganism culture before incubation and comparing the value with the optical density value obtained after incubation.

Thus, “independence of growth” of the microorganism according to the invention can be established by determining that a microorganism does not show growth in a specific medium and that said microorganism does still not show growth in said specific medium if at least one of the compounds selected from the group consisting of (i) a sulphide compound, (ii) methyl mercaptan, (iii) cadaverine, (iv) putrescine, (v) indole, and (vi) skatole is added.

The independence of growth of the microorganism according to the invention which is able to decrease the amount of least one of the compounds selected from the group consisting of (i) a sulphide compound, (ii) methyl mercaptan, (iii) cadaverine, (iv) putrescine, (v) indole, and (vi) skatole can preferably be observed in vitro, more preferably in an assay in which a microorganism according to the invention is cultivated in a nutrient-free buffer in the presence of odorous substances. The growth or absence of growth can be determined by photometrically measuring the optical density of a microorganism culture before incubation and comparing the value with the optical density value obtained after incubation.

Corresponding in vitro assays for growth monitoring are known to the person skilled in the art. An exemplary in vitro growth monitoring assay for microorganisms, preferably for lactic acid bacteria, is described herein below, or can preferably be derived from the Examples.

Briefly, such an assay may comprise the following steps:
mixing a microorganism which should be tested for its capability to decrease the amount of an odorous substances independent of its growth with a buffer and a solution of the odorous substance;
determination of optical density of mixture;
incubating the mixture under conditions allowing the decrease in amount of an odorous substance;
determination of optical density of the mixture after the incubation step;
extracting the supernatant of the mixture; and
detecting the amount of odorous substance in the supernatant.

The mixing of the components may be carried out in any suitable proportion and in any suitable buffer or medium, known to the person skilled in the art. In a preferred embodiment a microorganism which is able to decrease the amount of the odorous substance; is anaerobically cultivated in MRS broth at 37° C. The cultivation may be carried out, e.g., for 15 to 40 h, preferably for 20 to 35 h and even more preferably for 24 h. As volume for the anaerobic cultivation any volume suitable can be used, preferably a volume of 1 μl to 1 ml, more preferably 50 μl to 750 μl ml, even more preferably 100 to 300 μl, and most preferably 150 μl is used. The inoculation may be carried out by any mean known to the person skilled in the art. Preferably, an inoculum of a freezing culture is used. More preferably, 1 to 100 μl of a freezing culture are used, most preferably 10 μl of a freezing culture are used.

The microorganism which is able to decrease the amount of an odorous substance is subsequently separated from the culture medium by any suitable method, e.g. the culture of said microorganism can be centrifuged, for example at 4000 rpm for 15 min. As a further step the obtained microorganisms are washed by any suitable means known to the person skilled in the art, preferably an obtained cell pellet is washed one to several times in a buffer, e.g. a PBS-buffer, pH 7.0. As a further step, the obtained cells are resuspended in any suitable buffer, known to the person skilled in the art, preferably an obtained cell pellet is resuspended in, e.g. 150 μl of phosphate buffer, pH 7.0. Preferably, the buffer is a PBS buffer (10 mM phosphaste, 150 mM NaCl, pH 7.0).

For the assay cells of the microorganism, which is able to decrease the amount of odorous substances preferably washed cells, are mixed with an odorous substance, in any suitable proportion known to the person skilled in the art. In a preferred embodiment, 1 to 500 μl of washed cells are used, more preferably, 10 to 200 μl, even more preferably 30 to 100 μl and most preferably 50 μl are used. As a control any suitable buffer or medium instead of the cells, for instance, PBS-buffer or MRS medium in a suitable, corresponding amount may be added to the mixture as characterized herein above. The mixtures are measured by any means known to the person skilled in the art, leading to information on the amount and/or size of cells in the mixture. In a preferred embodiment, the measurement is carried out as determination of optical density of the mixture, even more preferably the optical density is measured photometrically at a wavelength of 600 nm.

The samples are subsequently incubated under conditions allowing the decrease of amount of the odorous substance. Such conditions are known to the skilled person. More preferably, the samples are incubated at 37° C. under anaerobic conditions, for example, for 1 min to 5 h, even more preferably 10 min to 3 h, 20 min to 2 h and most preferably for 1 h. Afterwards, the cells may be centrifuged.

After the incubation step the mixtures are again measured, leading to information on the amount and/or size of cells in the mixture. This measurement may be carried out by any means known to the person skilled in the art. In a preferred embodiment, the measurement is carried out as determination of optical density of the mixture, even more preferably the optical density is measured photometrically at a wavelength of 600 nm.

The presence of odorous compounds in the supernatant can be detected by methods known to the person skilled in the art. For example, the supernatant may be derivatised and further analyzed with any means known to the skilled artisan, e.g. as described herein above.

A microorganism is regarded as being able to reduce the generation of feces odor independent of the growth of the microorganism if the optical density of the mixture before the incubation step is not less than 70%, 80%, 90%, 95%, 96%, 97%, preferably not less than 98%, more preferably not less than 99% and most preferably not less than 100% of the optical density of the mixture after the incubation step.

The described assay may also be used to identify microorganisms, which are capable of reducing the generation of feces odor independent of the growth of the microorganism.

An exemplary in vitro growth monitoring assay for microorganisms, preferably for fungal or yeast cells, is described herein below, or can preferably be derived from the Examples.

Briefly, such an assay may comprise the following steps:
mixing a microorganism which should be tested for its capability to decrease the amount of an odorous substances independent of its growth with a buffer and a solution of the odorous substance;
determination of optical density of mixture;
incubating the mixture under conditions allowing the decrease in amount of an odorous substance;
determination of optical density of the mixture after the incubation step;
extracting the supernatant of the mixture; and
detecting the amount of odorous substance in the supernatant.

The mixing of the components may be carried out in any suitable proportion and in any suitable buffer or medium, known to the person skilled in the art. In a preferred embodiment a microorganism which is able to decrease the amount of the odorous substance; is aerobically cultivated in YM broth (Difco Manual; 3.0 g yeast extract, 3.0 g malt extract, 5.0 g peptone, 10.0 g dextrose per liter) at 30° C. The cultivation may be carried out, e.g., for 10 to 80 h, preferably for 20 to 60 h and even more preferably for 48 h. As volume for the aerobic cultivation any volume suitable can be used, preferably a volume of 1 μl to 1 ml, more preferably 50 μl to 750 μl ml, even more preferably 100 to 300 μl, and most preferably 150 μl is used.

The microorganism which is able to decrease the amount of an odorous substance is subsequently separated from the culture medium by any suitable method, e.g. the culture of said microorganism can be centrifuged, for example at 4000 rpm for 15 min. As a further step the obtained microorganisms are washed by any suitable means known to the person skilled in the art, preferably an obtained cell pellet is washed one to several times in a buffer, e.g. a PBS-buffer, pH 7.0. As a further step, the obtained cells are resuspended in any suitable buffer, known to the person skilled in the art, preferably an obtained cell pellet is resuspended in, e.g. 150 μl of phosphate buffer, pH 7.0. Preferably, the buffer is a PBS buffer (10 mM phosphate, 150 mM NaCl, pH 7.0).

For the assay cells of the microorganism, which is able to decrease the amount of odorous substances preferably washed cells, are mixed with an odorous substance, in any suitable proportion known to the person skilled in the art. In a preferred embodiment, 1 to 500 μl of washed cells are used, more preferably, 10 to 200 μl, even more preferably 30 to 100 μl and most preferably 50 μl are used. As a control any suitable buffer or medium instead of the cells, for instance, PBS-buffer or MRS medium in a suitable, corresponding amount may be added to the mixture as characterized herein above. The mixtures are measured by any means known to the person skilled in the art, leading to information on the amount and/or size of cells in the mixture. In a preferred embodiment, the measurement is carried out as determination of optical density of the mixture, even more preferably the optical density is measured photometrically at a wavelength of 600 nm.

The samples are subsequently incubated under conditions allowing the decrease of amount of the odorous substance. Such conditions are known to the skilled person. More preferably, the samples are incubated at 37° C. under anaerobic conditions, for example, for 1 min to 5 h, preferably 10 min to 3 h, more preferably 20 min to 2 h and most preferably for 1 h. Even more preferably, the samples may be shaken during the incubation. Afterwards, the cells may be centrifuged.

After the incubation step the mixtures are again measured, leading to information on the amount and/or size of cells in the mixture. This measurement may be carried out by any means known to the person skilled in the art. In a preferred embodiment, the measurement is carried out as determination of optical density of the mixture, even more preferably the optical density is measured photometrically at a wavelength of 600 nm.

The presence of odorous compounds in the supernatant can be detected by methods known to the person skilled in the art. For example, the supernatant may be derivatised and further analyzed with any means known to the skilled artisan, e.g. as described herein above.

A microorganism is regarded as being able to reduce the generation of feces odor independent of the growth of the microorganism if the optical density of the mixture before the incubation step is not less than 70%, 80%, 90%, 95%, 96%, 97%, preferably not less than 98%, more preferably not less than 99% and most preferably not less than 100% of the optical density of the mixture after the incubation step.

The described assay may also be used to identify microorganisms, which are capable of reducing the generation of feces odor independent of the growth of the microorganism.

The microorganism according to the invention may be resistant or sensitive to an antibiotic. The term “resistant to an antibiotic” means that the microorganism according to the invention is viable in the presence of an antibiotic. Preferably, the term means that the microorganism is able to grow under conditions, i.e. in the presence of an antibiotic, under which an organism sensitive to an antibiotic cannot grow. Such conditions are known to the person skilled in the art. Preferably, these conditions include the concentration of the antibiotic and the temperature of the incubation.

The term “sensitive to an antibiotic” means that the microorganism is inhibited in its growth or killed by an antibiotic. Preferably, the term means that the microorganism is not able to grow under conditions, i.e. in the presence of an antibiotic, under which an organism resistant to an antibiotic can grow. Such conditions are known to the person skilled in the art. Preferably, these conditions include the concentration of the antibiotic and the temperature of the incubation.

The term “antibiotic” refers to a chemical substance, which has the capacity to inhibit the growth of or to kill microorganisms. Such substances are known to the person skilled in the art. Preferably, the term refers to beta-lactam compounds like penicillines, cephalosporins or carbapenems; macrolides; tetracyclines; fluoroquinolones; sulphonamides; aminoglycosides; imidazoles; peptide-antibiotics and lincosamides. More preferably, the term relates to penicillin G, ampicillin, amoxicillin, flucloxacillin, methicillin, oxacillin, cefoxitin, ceftriaxone, ceftrizoxime, imipenem, erythromacin, tylosin, tilmicosin, spiramycin, josamycin, azithromycin, clarithromycin, tetracycline, minocycline, doxycycline, lymecycline, norfloxacin, ciprofloxacin, enoxacin, ofloxacin, co-trimoxazole, trimethoprim, gentamicin, amikacin, metronidazole, bactiracin, clindamycin or lincomycin. Most preferably, the term relates to ampicillin, cefotaxime, erythromycin, tetracycline, ciprofloxacin, co-trimoxazole, gentamicin, metronidazole, bacitracin or clindomycin.

The resistance or senstitivity to an antibiotic can be determinded by any means known to a person skilled in the art. Preferably, the resistance or sensitivity to an antibiotic may be tested in an assay for the lowest test concentration of the antibiotic which completely inhibits the growth of the microorganisms. In such an assay the antibiotic sensitivity of a microorganism may be regarded as the lowest test concentration of the antibiotic which completely inhibits the growth of the micoroorganism; i.e., Minimum Inhibitory Concentration or MIC. Antibiotic resistance of a microorganism may be regarded as the absence of a MIC for the antibiotic.

An exemplary method of testing for antibiotic sensitivity or resistance in a microorganism is described herein below, or can preferably be derived from the Examples.

Briefly such a testing for antibiotic sensitivity/resistance involves growth of a test microorgansims in the presence of various concentrations of the antibiotic of interest and is called the “disc method”. For the method any type of agar which is suitable, as known to the person skilled in the art, can be used. Preferably, an “iso-sensitest” agar may be used. As a next step, the agar surface may receive a suspension of a test microorganism, preferably any microorganism suitable, as known to the person skilled in the art, more preferable a bacterium. As quality control organisms any organisms suitable, as known to the person skilled in the art, may be used. The term “quality control organism” refers to an indicator strain which allows a comparison of the test results of the assay with the results of the assay with a known microorganism which has a known susceptibility to antibiotics. In a preferred embodiment such an assay may be carried out with a bacterium of the genus Staphylococcus, Escherichia or Pseudomonas as a quality control organism, most preferably such an assay is carried out with Staphylococcus aureus—ATCC 25923, Escherichia coli—ATCC 25922, or Pseudomonas aeruginosa—ATCC 27853 as a quality control organism. The suspension may then be spread out over the surface of the agar by any means known to the person skilled in the art so that a lawn of growing organisms can be obtained. Subsequently discs of an absorbent material may be added to the agar surface. In a preferred embodiment a plate may contain six discs. Each disc may be prepared by any suitable means know to the person skilled in the art. Preferably the disc may be soaked in a known and different concentration of the same or of a different antibiotics.

Subsequently the agar plates are incubated under suitable conditions known to the person skilled in the art. Preferably, the agar plates are incubated under conditions, which allow the growth of the test microorganisms. More preferably, the plates are incubated under conditions which allow the antibiotic to diffuse from each disc into the agar.

The agar plates may be analysed by any means known to the person skilled in the art. The concentration of the antibiotic is regarded to be lethal, if no growth of the test microorganism will occur. The concentration of the antibiotic is regarded to be below lethal concentration, if growth the test microorganism can occur. The results of the assay may be interpreted by any means suitable, as known to the person skilled in the art. In a preferred embodiment, the result is a ring of no growth around a disc. In a further preferred embodiment the result is the absence of a ring of no growth around a disc. The presence, size or diameter of the ring may then be interpreted by any means known to the person skilled in the art, e.g. by comparison with known standards. Preferably, the diameter of the growth inhibition ring may indicate at what minimal inhibitory concentration the test microorganism is sensitive to an antibiotic or, if no ring is observed, that the test microorganism is resistant to an antibiotic.

An further exemplary method of testing for antibiotic resistance and/or sensitivity in a microorganism via the Minimum Growth Concentration is described herein below, or can preferably be derived from the Examples.

Briefly such a testing for antibiotic sensitivity/resistance involves growth of a test microorgansims in the presence of various concentrations of the antibiotic of interest and is called the “agar plate method”. For the method any type of agar which is suitable, as known to the person skilled in the art, can be used. As a first step agar plates are prepared which contain different amounts of an antibiotic according to protocols known to the person skilled in the art. Preferably, the agar may be melted, cooled to 50° C.; mixed with an antibiotic with any suitable final concentration known to the person skilled in the art. Preferably, the antibiotic is added to a final concentration of, e.g. 0 μg/ml, 0.1 μg/ml, 0.2 μg/ml 0.4 μg/ml, 1.0 μg/ml, 2.0 μg/ml, 4.0 μg/ml, 6.0 μg/ml, 8.0 μg/ml and 10.0 μg/ml.

Subsequently, the plates may be dried, and each plate may be divided, e.g., into eight sectors with a marker on the back of the plate. A test microorganism, preferably any microorganism suitable, as known to the person skilled in the art, more preferable a bacterium may be cultivated according to suitable protocols, known to the person skilled in the art. As quality control organisms any organisms suitable, as known to the person skilled in the art, may be used. In a preferred embodiment such an assay may be carried out with a bacterium of the genus Staphylococcus, Escherichia or Pseudomonas as a quality control organism, most preferably such an assay is carried out with Staphylococcus aureus—ATCC 25923, Escherichia coli—ATCC 25922, or Pseudomonas aeruginosa—ATCC 27853 as a quality control organism. The test microorganism may be cultivated overnight in a suitable medium known to the person skilled in the art. Subsequently a dilution of each culture may be prepared according to suitable protocols known to the skilled person. Preferably, a dilution of each culture may be prepared by adding the overnight broth culture to 1 ml of saline until the turbidity approximately matches that of a McFarland 0.5 nephelometry standard. Subsequently, the suspension of the test microorganisms may be applied to the agar plate by any means known to the person skilled in the art. Preferably, a sterile cotton-tipped applicator may be dipped into the test microorganism suspension and the excess fluid may be squeezed out against the inside of the tube. Subsequently, a single radial streak of an inch in length may be made to the corresponding sector of each plate of the series, beginning with the control plate (no antibiotic) and progressing through the increasing concentration plates. After the inocula have dried or have been absorbed into the agar plate medium the plates may be closed and incubated under suitable conditions known to the person skilled in the art. Preferably, the plates may be incubated for 24 h at 35° C. The agar plates may be analysed by any means known to the person skilled in the art. Preferably, growth may be observed and recorded using the following scale: growth equivalent to control ++++; moderate growth +++; intermediate growth ++; scant growth +; no growth −. Preferably, growth pattern may indicate at what minimal inhibitory concentration the test microorganism is sensitive to an antibiotic or whether the test microorganism is resistant to an antibiotic. More preferably, the minimal inhibitory concentration may be regarded as the lowest concentration of the antibiotic tested that yields complete inhibition of growth.

The term “microorganism” refers to a minute, microscopic or submicroscopic living organisms. Preferably the term includes bacteria, fungi, and protozoa.

In a particularly preferred embodiment the microorganism of the present invention is a microorganism belonging to the group of lactic acid bacteria. The term “microorganism belonging to the group of lactic acid bacteria” encompasses (a) microorganism(s) which belong(s) to bacteria, in particular belonging to gram-positive fermentative eubacteria, more particularly belonging to the family of lactobacteriaceae including lactic acid bacteria. Lactic acid bacteria are from a taxonomical point of view divided up into the subdivisions of Streptococcus, Leuconostoc, Pediococcus and Lactobacillus. The microorganism of the present invention is preferably a Lactobacillus species. Members of the lactic acid bacteria group normally lack porphyrins and cytochromes, do not carry out electron-transport phosphorylation and hence obtain energy only by substrate-level phosphorylation. I.e. in lactic acid bacteria ATP is synthesized through fermentation of carbohydrates. All of the lactic acid bacteria grow anaerobically, however, unlike many anaerobes, most lactic acid bacteria are not sensitive to oxygen and can thus grow in its presence as well as in its absence. Accordingly, the bacteria of the present invention are preferably aerotolerant anaerobic lactic acid bacteria, preferably belonging to the genus of Lactobacillus.

The lactic acid bacteria of the present invention are preferably rod-shaped or spherical, varying from long and slender to short bent rods, are moreover preferably immotile and/or asporogenous and produce lactic acid as a major or sole product of fermentative metabolism. The genus Lactobacillus to which the microorganism of the present invention belongs in a preferred embodiment is divided up by the following characteristics into three major subgroups, whereby it is envisaged that the Lactobacillus species of the present invention can belong to each of the three major subgroups:

(a) homofermentative lactobacilli
(i) producing lactic acid, preferably the L-, D- or DL-isomer(s) of lactic acid in an amount of at least 85% from glucose via the Embden-Meyerhof pathway;
(ii) growing at a temperature of 45° C., but not at a temperature of 15° C.;
(iii) being long-rod shaped; and
(iv) having glycerol teichoic acid in the cell wall;
   (b) homofermentative lactobacilli
(i) producing lactic acid, preferably the L- or DL-isomer(s) of lactic acid via the Embden-Meyerhof pathway;
(ii) growing at a temperature of 15° C., showing variable growth at a temperature of 45° C.;
(iii) being short-rod shaped or coryneform; and
(iv) having ribitol and/or glycerol teichoic acid in their cell wall;
   (c) heterofermentative lactobacilli
(i) producing lactic acid, preferably the DL-isomer of lactic acid in an amount of at least 50% from glucose via the pentose-phosphate pathway;
(ii) producing carbondioxide and ethanol
(iii) showing variable growth at a temperature of 15° C. or 45° C.;
(iv) being long or short rod shaped; and
(v) having glycerol teichoic acid in their cell wall.

Based on the above-described characteristics, the microorganisms of the present invention can be classified to belong to the group of lactic acid bacteria, particularly to the genus of Lactobacillus. By using classical systematics, for example, by reference to the pertinent descriptions in “Bergey's Manual of Systematic Bacteriology” (Williams & Wilkins Co., 1984), a microorganism of the present invention can be determined to belong to the genus of Lactobacillus. Alternatively, the microorganisms of the present invention can be classified to belong to the genus of Lactobacillus by methods known in the art, for example, by their metabolic fingerprint, i.e. a comparable overview of the capability of the microorganism(s) of the present invention to metabolize sugars or by other methods described, for example, in Schleifer et al., System. Appl. Microb., 18 (1995), 461-467 or Ludwig et al., System. Appl. Microb., 15 (1992), 487-501. The microorganisms of the present invention are capable of metabolizing sugar sources which are typical and known in the art for microorganisms belonging to the genus of Lactobacillus.

The affiliation of the microorganisms of the present invention to the genus of Lactobacillus can also be characterized by using other methods known in the art, for example, using SDS-PAGE gel electrophoresis of total protein of the species to be determined and comparing them to known and already characterized strains of the genus Lactobacillus. The techniques for preparing a total protein profile as described above, as well as the numerical analysis of such profiles, are well known to a person skilled in the art. However, the results are only reliable insofar as each stage of the process is sufficiently standardized. Faced with the requirement of accuracy when determining the attachment of a microorganism to the genus of Lactobacillus, standardized procedures are regularly made available to the public by their authors such as that of Pot et al., as presented during a “workshop” organized by the European Union, at the University of Ghent, in Belgium, on Sep. 12 to 16, 1994 (Fingerprinting techniques for classification and identification of bacteria, SDS-PAGE of whole cell protein). The software used in the technique for analyzing the SDS-PAGE electrophoresis gel is of crucial importance since the degree of correlation between the species depends on the parameters and algorithms used by this software. Without going into the theoretical details, quantitative comparison of bands measured by a densitometer and normalized by a computer is preferably made with the Pearson correlation coefficient. The similarity matrix thus obtained may be organized with the aid of the UPGMA (unweighted pair group method using average linkage) algorithm that not only makes it possible to group together the most similar profiles, but also to construct dendograms (see Kersters, Numerical methods in the classification and identification of bacteria by electrophoresis, in Computer-assisted Bacterial Systematics, 337-368, M. Goodfellow, A. G. O'Donnell Ed., John Wiley and Sons Ltd, 1985).

Alternatively, the affiliation of said microorganisms of the present invention to the genus of Lactobacillus can be characterized with regard to ribosomal RNA in a so called Riboprinter®. More preferably, the affiliation of the newly identified species of the invention to the genus Lactobacillus is demonstrated by comparing the nucleotide sequence of the 16S ribosomal RNA of the bacteria of the invention, or of their genomic DNA which codes for the 16S ribosomal RNA, with those of other genera and species of lactic acid bacteria known to date. Another preferred alternative for determining the attachment of the newly identified species of the invention to the genus Lactobacillus is the use of species-specific PCR primers that target the 16S-23S rRNA spacer region. Another preferred alternative is RAPD-PCR (Niaatu et al. in Antonie van Leenwenhoek (79), 1-6, 2001) by virtue of that a strain specific DNA pattern is generated which allows to determine the affiliation of an identified microorganisms in accordance with the present invention to the genus of Lactobacillus. Further techniques useful for determining the affiliation of the microorganism of the present invention to the genus of Lactobacillus are restriction fragment length polymorphism (RFLP) (Giraffa et al., Int. J. Food Microbiol. 82 (2003), 163-172), fingerprinting of the repetitive elements (Gevers et al., FEMS Microbiol. Lett. 205 (2001) 31-36) or analysis of the fatty acid methyl ester (FAME) pattern of bacterial cells (Hevrman et al., FEMS Microbiol. Lett. 181 (1991), 55-62). Alternatively, lactobacilli can be determined by lectin typing (Annuk et al., J. Med. Microbiol. 50 (2001), 1069-1074) or by analysis of their cell wall proteins (Gatti et al., Lett. Appl. Microbiol. 25 (1997), 345-348.

In a further particularly preferred embodiment the microorganism of the present invention is a microorganism belonging to the group of yeasts. The term “microorganism belonging to the group of yeast” encompasses (a) microorganism(s) which belong(s) to eukaryotic microorganisms, in particular belonging to single-celled (unicellular) fungi, more particularly belonging to the families of ascomycota and basidiomycota. Members of the group of yeasts are heterotrophic, lack chlorophyll, and are characterized by a wide dispersion of natural habitats. Yeasts are common on plant leaves and flowers, soil and salt water and are especially abandoned in sugar mediums such as flower nectar and fruits. Yeasts are also found on the skin surfaces and in the intestinal tracts of warm-blooded animals, where they may live symbiotically or as parasites. Yeasts multiply as single cells that divide by budding or direct division (fission), or they may grow as simple irregular filaments (mycelium).

Many of one subdivision of the yeasts, the ascomycota, consist of hyphae, i.e. long thin thread-shaped cells approximately 5 μm thick which form the mycelium, a woolly interlaced mesh. A group of species of the ascomycota are dimorphic, which means that they can appear either in single- or multi-cellular form.

The cell walls of ascomycota are almost always formed of chitin and β-glucans; individual cells are divided by septa. These give stability to the hyphae and prevent a loss of cytoplasm in the event that the cell membrane should be locally damaged. As a result ascomycetes can live in dry environments. Mostly the cell divisions are centrally perforated, so they have a small opening in the middle, through which cytoplasm and also nuclei can move more or less freely throughout the system of hyphae. Most hyphae only have one nucleus per cell, and are therefore described as uninucleate.

Ascomycota fulfil a central role in most land-based ecosystems. They are important decomposers which break down such organic materials as dead leaves, twigs, fallen trees, etc. and help the detritivores (animals which live off this decomposing material) to obtain their nutrients. By processing substances like cellulose or lignin, which are otherwise difficult to exploit, they take on an important place in the natural nitrogen cycle and the carbon cycle.

The ascomycota principally digest living or dead biomass. To achieve this, they excrete into their surroundings digestive enzymes which break down organic substances, which are then absorbed through the cell wall. Many species live on dead plant material such as fallen leaves, twigs, or indeed large logs. Others attack plants, animals, or other fungi as parasites and derive their metabolic energy, as well as all the nutrients they need, from the cell tissue of their hosts. In the course of their evolutionary history the ascomycota have achieved the capability of breaking down almost every organic substance. They are able to digest with their own enzymes plant cellulose and the lignin contained in wood. Also collagen and keratin serve as food sources. Examples of yeast genera belonging to the group of ascomycota are Saccharomyces, Saccharomycopsis, Saccharomycodes, Schizosaccharomyces, Wickerhamia, Debaryomyces, Hansenula, Hanseniaspora, Pichia, Kloeckera, Candida, Zygosaccharomyces, Ogataea, Kuraishia, Komagataella, Yarowia, Metschnikowia, Williopsis, Nakazawaea, Kluyveromyces, and Torulaspora. In a preferred embodiment the microorganism of the present invention belongs to the genus Kluyveromyces, Candida or Metschnikowia.

The second subdivision of yeasts, the basidiomycota, includes species that produce spores in a club-shaped structure called a basidium. The basidiomycota is thought to comprise three major clades, the hymenomycotina (Hymenomycetes; mushrooms), the ustilaginomycotina (Ustilaginomycetes; true smut fungi), and the teliomycotina (Urediniomycetes; rusts). Basidiomycota include both unicellular and multicellular forms and sexual and asexual species. They occur in terrestrial and aquatic environments (including the marine environment) and can be characterized by bearing sexual spores on basidia, having a long-lived dikaryon, and usually showing clamp connections.

Examples of yeast genera belonging to the group of basidiomycota are Cryptococcus, Bullera, Rhodotorula and Sporobolomyces. In a preferred embodiment the microorganism of the present invention belongs to the genus Cryptococcus.

By using classical systematics, for example, by reference to the pertinent descriptions in “The yeasts” (N. J. W. Kreger-van Rij, 1984) or “Yeasts” (Barnet, Payne and Yarrow, 1990) a microorganism of the present invention can be determined to belong to the group of yeasts. Alternatively, the microorganisms of the present invention can be classified to belong to the group of yeasts by methods known in the art, for example, by macroscopic and microscopic appearance, formation of mycelia, formation of spores, fermentation of different substrates, assimilation of different substrates or growth on inhibitory substances or by any other method known to the skilled person or described, for example, in “The yeasts” (N. J. W. Kreger-van Rij, 1984) or “Yeasts” (Barnet, Payne and Yarrow, 1990).

The affiliation of the microorganisms of the present invention to the group of yeasts and the further systematic identification and elucidation of said microorganisms of the present invention can also be achieved by using other methods known in the art, for example, rRNA analysis. Preferably genes that encode the rRNA, i.e. rDNA genes, may be sequenced in order to characterize an organism's taxonomic situation, for example, by calculating related taxonomic groups and estimating rates of species divergence. In a preferred embodiment a 18S rRNA analysis as described, e.g., in Takashima et al. (Intl J Syst Evol Microbiol, 50, 3 (2000), 1351-1371) may be used to elucidate the systematic or taxonomic situation of a microorganism of the invention. In a further preferred embodiment a 26S rRNA analysis as described, e.g., in Chen et al. (J Clin Microbiol, 39, 11 (2001), 4042-4051) by virtue of the identification of a polymorphic internal transcribed spacer region 1 of the 26S rRNA may be used to elucidate the systematic or taxonomic situation of a microorganism of the invention. Another preferred alternative for determining the taxonomical situation of the newly identified yeast species of the invention is the use of a rDNA (D1/D2 domain) analysis as described in Fell et al. (Int J Syst Evol Microbiol, 51, 3 (2000), 1351-1371) which is based on differences in the large subunit rDNA D1/D2 domain sequences. A further technique useful for determining the taxonomical situation of the yeast species of the invention is molecular fingerprinting as described, for example, in Neppelenbroek et al. (Oral Dis, 12, 3 (2006), 242-253).

In a preferred embodiment of the present application the microorganism is a probiotic Lactobacillus or yeast species. The term “probiotic” in the context of the present invention means that the microorganism has a beneficial effect on health if it is ingested. Preferably, a “probiotic” microorganism is a live microorganism which, when ingested, is beneficial for health of the gastrointestinal tract. Most preferably, this means that the microorganism has a positive effect on the micro flora of the gastrointestinal tract.

In a preferred embodiment the microorganism of the present invention belongs to the species of Lactobacillus paracasei ssp. paracasei, Lactobacillus rhamnosus, Lactobacillus plantarum, Lactobacillus crispatus, Lactobacillus acidophilus, Lactobacillus delbrückii ssp. delbrückii or Lactoabacillus curvatus. However, the Lactobacillus species are not limited thereto.

In a particularly preferred embodiment of the present invention the microorganism of the present invention is selected from the group consisting of Lactobacillus paracasei ssp. paracasei, Lactobacillus rhamnosus, Lactobacillus acidophilus, Lactobacillus plantarum, Lactobacillus crispatus, Lactobacillus delbrückii ssp. delbrückii and Lactoabacillus curvatus being deposited at the DSMZ under the accession number DSM 18456 (Lactobacillus paracasei ssp. paracasei GU-Lb-0001), DSM 18457 (Lactobacillus rhamnosus GU-Lb-0002), DSM 18458 (Lactobacillus acidophilus GU-Lb-0003), DSM 18459 (Lactobacillus acidophilus GU-Lb-0004), DSM 18460 (Lactobacillus rhamnosus GU-Lb-0005), DSM 18461 (Lactobacillus acidophilus GU-Lb-0006), DSM 18462 (Lactobacillus acidophilus GU-Lb-0007), DSM 18463 (Lactobacillus paracasei ssp. paracasei GU-Lb-0008), DSM 18464 (Lactobacillus crispatus GU-Lb-0009), DSM 18465 (Lactobacillus delbrückii ssp. delbrückii GU-Lb-0010), DSM 18466 (Lactobacillus curvatus GU-Lb-0011), DSM 18467 (Lactobacillus crispatus GU-Lb-0012), DSM 18468 (Lactobacillus plantarum GU-Lb-0013), DSM 18469 (Lactobacillus acidophilus GU-Lb-0014) and DSM 18470 (Lactobacillus acidophilus GU-Lb-0015).

The term “Lactobacillus paracasei ssp. paracasei, Lactobacillus rhamnosus, Lactobacillus plantarum, Lactobacillus crispatus, Lactobacillus acidophilus, Lactobacillus delbrückii ssp. delbrückii and Lactoabacillus curvatus being deposited at the DSMZ under the accession number” relates to cells of a microorganism belonging to the species Lactobacillus paracasei ssp. paracasei, Lactobacillus rhamnosus, Lactobacillus plantarum, Lactobacillus crispatus, Lactobacillus acidophilus, Lactobacillus delbrückii ssp. delbrückii or Lactoabacillus curvatus deposited at the Deutsche Sammlung für Mikroorganismen and Zellkulturen (DSMZ) on Jul. 13, 2006 and having the following deposit numbers: DSM 18456 (Lactobacillus paracasei ssp. paracasei GU-Lb-0001), DSM 18457 (Lactobacillus rhamnosus GU-Lb-0002), DSM 18458 (Lactobacillus acidophilus GU-Lb-0003), DSM 18459 (Lactobacillus acidophilus GU-Lb-0004), DSM 18460 (Lactobacillus rhamnosus GU-Lb-0005), DSM 18461 (Lactobacillus acidophilus GU-Lb-0006), DSM 18462 (Lactobacillus acidophilus GU-Lb-0007), DSM 18463 (Lactobacillus paracasei ssp. paracasei GU-Lb-0008), DSM 18464 (Lactobacillus crispatus GU-Lb-0009), DSM 18465 (Lactobacillus delbrückii ssp. delbrückii GU-Lb-0010), DSM 18466 (Lactobacillus curvatus GU-Lb-0011), DSM 18467 (Lactobacillus crispatus GU-Lb-0012), DSM 18468 (Lactobacillus plantarum GU-Lb-0013), DSM 18469 (Lactobacillus acidophilus GU-Lb-0014) and DSM 18470 (Lactobacillus acidophilus GU-Lb-0015). The DSMZ is located at the Mascheroder Weg 1b, D-38124 Braunschweig, Germany. The aforementioned deposits were made pursuant to the terms of the Budapest treaty on the international recognition of the deposit of microorganisms for the purposes of patent procedures.

In another preferred embodiment the microorganism of the present invention belongs to the yeast species of Cryptococcus laurentii, Kluyveromyces marxianus, Candida haemulonii or Metschnikowia reukaufii.

However, the yeast species are not limited thereto.

In a particularly preferred embodiment of the present invention the microorganism of the present invention is selected from the group consisting of Cryptococcus laurentii, Kluyveromyces marxianus, Candida haemulonii and Metschnikowia reukaufii being deposited at the DSMZ under the accession number DSM 18471 (Cryptococcus laurentii GU-Ye-0001), DSM 18472 (Kluyveromyces marxianus GU-Ye-0002), DSM 18473 (Candida haemulonii GU-Ye-0003) and DSM 18474 (Metschnikowia reukaufii GU-Ye-0004).

The term “Cryptococcus laurentii, Kluyveromyces marxianus, Candida haemulonii and Metschnikowia reukaufii being deposited at the DSMZ under the accession number” relates to cells of a microorganism belonging to the species Cryptococcus laurentii, Kluyveromyces marxianus, Candida haemulonii or Metschnikowia reukaufii deposited at the Deutsche Sammlung für Mikroorganismen and Zellkulturen (DSMZ) on Jul. 13, 2006 and having the following deposit numbers: DSM 18471 (Cryptococcus laurentii GU-Ye-0001), DSM 18472 (Kluyveromyces marxianus GU-Ye-0002), DSM 18473 (Candida haemulonii GU-Ye-0003) and DSM 18474 (Metschnikowia reukaufii GU-Ye-0004). The DSMZ is located at the Mascheroder Weg 1b, D-38124 Braunschweig, Germany. The aforementioned deposits were made pursuant to the terms of the Budapest treaty on the international recognition of the deposit of microorganisms for the purposes of patent procedures.

In a particular preferred embodiment the microorganisms of the present invention are “isolated” or “purified”. The term “isolated” means that the material is removed from its original environment, e.g. the natural environment if it is naturally occurring, or the culture medium if it is cultured. For example, a naturally-occurring microorganism, preferably a Lactobacillus or yeast species, separated from some or all of the coexisting materials in the natural system, is isolated. Such a microorganism could be part of a composition, and is to be regarded as still being isolated in that the composition is not part of its natural environment.

The term “purified” does not require absolute purity; rather, it is intended as a relative definition. Individual microorganisms obtained from a library have been conventionally purified to microbiological homogeneity, i.e. they grow as single colonies when streaked out on agar plates by methods known in the art. Preferably, the agar plates that are used for this purpose are selective for Lactobacillus or yeast species. Such selective agar plates are known in the art.

In another aspect the present invention relates to an inactivated form of the microorganism of the present invention, which is, e.g., thermally inactivated or lyophilized, but which retains the ability to decrease the amount of a sulphide compound, methyl mercaptan, cadaverine, putrescine, indole or skatole.

According to the present invention the term “inactivated form of the microorganism of the present invention” includes a dead or inactivated cell of the microorganism of the present invention, preferably of the Lactobacillus or yeast species disclosed herein, which is no longer capable to form a single colony on a plate specific for microorganisms belonging to the genus of Lactobacillus or to the yeasts. Said dead or inactivated cell may have either an intact or broken cell membrane. Methods for killing or inactivating cells of the microorganism of the present invention are known in the art. El-Nezami et al., J. Food Prot. 61 (1998), 466-468 describes a method for inactivating Lactobacillus species by UV-irradiation and Kim et al., Photochem. Photobiol 79(4) (2004), 349-355 describes the inactivation of yeast species using UV-light radiation and heat.

Preferably, the cells of the microorganism of the present invention are thermally inactivated or lyophilised. Lyophilisation of the cells of the present invention has the advantage that they can be easily stored and handled while retaining their ability to decrease the amount of a sulphide compound, methyl mercaptan, cadaverine, putrescine, indole or skatole. Moreover, lyophilised cells can be grown again when applied under conditions known in the art to appropriate liquid or solid media. Lyophilization is done by methods known in the art. Preferably, it is carried out for at least 2 hours at room temperature, i.e. any temperature between 16° C. and 25° C. Moreover, the lyophilized cells of the microorganism of the present invention are stable for at least 4 weeks at a temperature of 4° C. so as to still retain their properties as described above. Thermal inactivation can be achieved by incubating the cells of the microorganism of the present invention for at least 2 hours at a temperature of 170° C. Yet, thermal inactivation is preferably achieved by autoclaving said cells at a temperature of 121° C. for at least 20 minutes in the presence of satured steam at an atmospheric pressure of 2 bar. In the alternative, thermal inactivation of the cells of the microorganism of the present invention is achieved by freezing said cells for at least 4 weeks, 3 weeks, 2 weeks, 1 week, 12 hours, 6 hours, 2 hours or 1 hour at −20° C. It is preferred that at least 70%, 75% or 80%, more preferably 85%, 90% or 95% and particularly preferred at least 97%, 98%, 99% and more particularly preferred, 99.1%, 99.2%, 99.3%, 99.4%, 99.5%, 99.6%, 99.7%, 99.8% or 99.9% and most particularly preferred 100% of the cells of the inactivated form of the microorganism of the present invention are dead or inactivated, however, they have still the ability to decrease the amount of a sulphide compound, methyl mercaptan, cadaverine, putrescine, indole or skatole.

Whether the inactivated form of the microorganism of the present invention is indeed dead or inactivated can be tested by methods known in the art, for example, by a test for viability.

The term “inactivated form of the microorganism of the present invention” also encompasses lysates or fractions of the microorganism of the present invention, preferably of the Lactobacillus or yeast species disclosed herein, wherein said lysates or fractions preferably have the ability to decrease the amount of a sulphide compound, methyl mercaptan, cadaverine, putrescine, indole or skatole. This ability can be tested as described herein and in particular as described in the appended Examples. In case, a lysate or fraction of the microorganism of the present invention may have the ability to decrease the amount of a sulphide compound, methyl mercaptan, cadaverine, putrescine, indole or skatole, then the skilled person can, for example, further purify said lysate or fraction by methods known in the art, which are exemplified herein below, so as to remove substances which may interfere with said ability. Afterwards the person skilled in the art can again test said lysate or fraction whether it has the ability to decrease the amount of a sulphide compound, methyl mercaptan, cadaverine, putrescine, indole or skatole.

According to the present invention the term “lysate” means a solution or suspension in an aqueous medium of cells of the microorganism of the present invention that are broken or an extract. However, the term should not be construed in any limiting way. The cell lysate comprises, e.g., macromolecules, like DNA, RNA, proteins, peptides, carbohydrates, lipids and the like and/or micromolecules, like amino acids, sugars, lipid acids and the like, or fractions of it. Additionally, said lysate comprises cell debris which may be of smooth or granular structure. Methods for preparing cell lysates of microorganism are known in the art, for example, by employing French press, cells mill using glass or iron beads or enzymatic cell lysis and the like. In addition, lysing cells relates to various methods known in the art for opening/destroying cells. The method for lysing a cell is not important and any method that can achieve lysis of the cells of the microorganism of the present invention may be employed. An appropriate one can be chosen by the person skilled in the art, e.g. opening/destruction of cells can be done enzymatically, chemically or physically. Non-limiting examples for enzymes and enzyme cocktails are proteases, like proteinase K, lipases or glycosidases; non-limiting examples for chemicals are ionophores, detergents, like sodium dodecyl sulfate, acids or bases; and non-limiting examples of physical means are high pressure, like French-pressing, osmolarity, temperature, like heat or cold. Additionally, a method employing an appropriate combination of an enzyme other than the proteolytic enzyme, an acid, a base and the like may also be utilized. For example, the cells of the microorganism of the present invention are lysed by freezing and thawing, more preferably freezing at temperatures below −70° C. and thawing at temperatures of more than 30° C., particularly freezing is preferred at temperatures below −75° C. and thawing is preferred at temperatures of more than 35° C. and most preferred are temperatures for freezing below −80° C. and temperatures for thawing of more than 37° C. It is also preferred that said freezing/thawing is repeated for at least 1 time, more preferably for at least 2 times, even more preferred for at least 3 times, particularly preferred for at least 4 times and most preferred for at least 5 times.

Accordingly, those skilled in the art can prepare the desired lysates by referring to the above general explanations, and appropriately modifying or altering those methods, if necessary. Preferably, the aqueous medium used for the lysates as described is water, physiological saline, or a buffer solution. An advantage of a bacterial or yeast cell lysate is that it can be easily produced and stored cost efficiently since less technical facilities are needed.

According to the invention, lysates are also preparations of fractions of molecules from the above-mentioned lysates. These fractions can be obtained by methods known to those skilled in the art, e.g., chromatography, including, e.g., affinity chromatography, ion-exchange chromatography, size-exclusion chromatography, reversed phase-chromatography, and chromatography with other chromatographic material in column or batch methods, other fractionation methods, e.g., filtration methods, e.g., ultrafiltration, dialysis, dialysis and concentration with size-exclusion in centrifugation, centrifugation in density-gradients or step matrices, precipitation, e.g., affinity precipitations, salting-in or salting-out (ammoniumsulfate-precipitation), alcoholic precipitations or other proteinchemical, molecular biological, biochemical, immunological, chemical or physical methods to separate above components of the lysates. In a preferred embodiment those fractions which are more immunogenic than others are preferred. Those skilled in the art are able to choose a suitable method and determine its immunogenic potential by referring to the above general explanations and specific explanations in the examples herein, and appropriately modifying or altering those methods, if necessary.

Accordingly, the term “an inactive form of the microorganism of the present invention” also encompasses filtrates of the microorganism of the present invention, preferably of the Lactobacillus or yeast species disclosed herein, wherein said filtrates preferably have the ability to decrease the amount of a sulphide compound, methyl mercaptan, cadaverine, putrescine, indole or skatole. This inhibition can be tested as described herein and in particular as described in the appended Examples. In case, a filtrate of the microorganism of the present invention may not decrease the amount of a sulphide compound, methyl mercaptan, cadaverine, putrescine, indole or skatole, then the skilled person can, for example, further purify said filtrate by methods known in the art, so as to remove substances which may inhibit the decrease. Afterwards the person skilled in the art can again test said filtrate whether it decreases the amount of a sulphide compound, methyl mercaptan, cadaverine, putrescine, indole or skatole.

The term “filtrate” means a cell-free solution or suspension of the microorganism of the present invention which has been obtained as supernatant of a centrifugation procedure of a culture of the microorganism of the present invention in any appropriate liquid, medium or buffer known to the person skilled in the art. However, the term should not be construed in any limiting way. The filtrate comprises, e.g., macromolecules, like DNA, RNA, proteins, peptides, carbohydrates, lipids and the like and/or micromolecules, like amino acids, sugars, lipid acids and the like, or fractions of it. Methods for preparing filtrates of microorganism are known in the art. In addition, “filtrate” relates to various methods known in the art. The exact method is not important and any method that can achieve filtration of the cells of the microorganism of the present invention may be employed.

The term “an inactive form of the microorganism of the present invention” encompasses any part of the cells of the microorganism of the present invention. Preferably, said inactive form is a membrane fraction obtained by a membrane-preparation. Membrane preparations of microorganisms belonging to the genus of Lactobacillus can be obtained by methods known in the art, for example, by employing the method described in Rollan et al., Int. J. Food Microbiol. 70 (2001), 303-307, Matsuguchi et al., Clin. Diagn. Lab. Immunol. 10 (2003), 259-266 or Stentz et al., Appl. Environ. Microbiol. 66 (2000), 4272-4278 or Varmanen et al., J. Bacteriology 182 (2000), 146-154. Alternatively, a whole cell preparation is also envisaged.

In another aspect the present invention relates to a composition comprising a microorganism according to the present invention or a mutant, derivative or inactive form of this microorganism as described above. In a preferred embodiment, said composition comprises either any microorganism of the invention alone or any combination of the microorganisms of the invention. In a preferred embodiment, said composition comprises a microorganism or combination of microorgansims as described above in an amount between 10² to 10¹² cells, preferably 10³ to 10¹⁰ cells per mg in a solid form of the composition. In case of a liquid form of compositions, the amount of the microorganisms is between 10² to 10¹³ cells per ml. In a further preferred embodiment said compositions are in the form of pellets, spray-dried powders, agglomerates, granulates, extrudates or compactates.

In case of pellets, spray-dried powders, agglomerates, granulates, extrudates or compactates. The compositions comprise a microorganism or combination of microorganisms as described herein in an amount between 10² to 10¹³ cells per ml. However, for specific compositions the amount of the microorganism may be different as is described herein.

The term “composition”, as used in accordance with the present invention, relates to (a) composition(s) which comprise(s) at least one microorganism of the present invention or mutant, derivative or inactive form of said microorganism as described above. The term “composition” also refers to any combination of microorganisms of the invention. It is envisaged that the compositions of the present invention which are described herein below comprise the aforementioned components in any combination. It may, optionally, comprise at least one further ingredient suitable for reducing the generation of feces odor. Accordingly, it may optionally comprise any combination of the hereinafter described further ingredients.

The composition may be in solid, liquid or gaseous form and may be, inter alia, in the form of (a) powder(s), (a) spray-dried powder(s), (a) tablet(s), (a) solution(s), (an) aerosol(s), granules, pills, suspensions, emulsions, capsules, syrups, liquids, elixirs, extracts, tincture, fluid extracts, (a) pellet(s), agglomerates, granulates, extrudates or compactates or in a form which is particularly suitable for oral administration or direct application. Preferably, the composition may be used as dry formulation (for mammalian and avian species) before pelleting or added in liquid form after pelleting (post-pelleting).

The dry composition may be produced by processes known in the art, preferably by changing the dry substance content (e.g. by drying or evaporation), grinding and formulation (e.g. addition of additives, shaping processes such as pelleting and extrusion). Furthermore, the processing of the by-product may also comprise mixing with other ingredients like animal feeds and feed additives, e.g. for standardizing the nutrient content. Drying processes are knonw to the person skilled in the art and disclosed, e.g., in O. Krischer, W. Kast: Die wissenschaftlichen Grundlagen der Trocknungstechnik, 3^{rd} Edition, Springer, Berlin-Heidelberg-New York 1978; R. B. Keey: Drying: Principles and Practice, Pergamon Press, Oxford 1972; K. Kröll: Trockner und Trocknungsverfahren, 2^{nd} Edition, Springer, Berlin-Heidelberg-New York 1978; Williams-Gardener, A.: Industrial Drying, Houston, Gulf, 1977; K. Kröll W. Kast: Trocknen und Trockner in der Produktion, Springer, Berlin-Heidelberg-New York 1989. Examples for drying processes include convective drying processes, e.g. in a kiln, tunnel dryer, conveyor dryer, disk dryer, jet dryer, fluidized bed dryer, vented as well as rotary drum dryers, spray dryer, flow type dryer, cyclone dryer, mixer dryer, micro grinding dryer, grinding dryer, ring dryer, column dryer, rotary dryer (tubular type), carousel dryer. Further processes may make use of contact drying, e.g paddle dryer; vacuum drying or lyophilization, conical dryer, Nutsche filter dryer, disk dryer, thin-layer contact dryer, drum dryer, viscosity phase, slurry dryer, plate dryer, spiral conveyor dryer, double cone dryer; or thermal radiation (infrared, e.g. infrared rotary dryer) or dielectric energy (microwaves) for drying. The drying apparatuses used for thermal drying processes may mostly be heated by vapor, oil, gas or electric current, and may be, depending on their construction, partly be operated under vacuum.

Formulation processes other than drying may used as described further below for the preparation of the protein composition. This includes also, inter alia, the addition of formulation auxiliaries, such as carrier and coating materials, binders and other additives.

The composition in the form of sprays or spray-dried powder, may be obtained in a process for preparing dry powder, preferably in a process in which the product is prepared and the whole drying process is carried out at significantly lower temperatures than with spray drying, usually at temperatures in the range from 10-70° C. Usually, drying takes from 1 to 10 hours. Spray formulation may be carried out in the presence of a pulverizing agent, e.g. hydrophobic silica or starch. This process is, for instance, described in EP 74050 and EP 285682. In a preferred embodiment a ready-to-use solution with adjusted viscosity (or solids content) may be sprayed in a tower in a cloud of the pulverizing agent and subsequently be dried on a fluid bed with an adjusted temperature-time profile.

By adding formulation auxiliaries, such as carrier and coating materials, binders and other additives, the properties of the dried by-product (i.e. the protein composition), present together with the solid fermentation components, may be selectively confectioned in a manner known to the person skilled in the art with regard to various parameters, such as grain size, particle form, propensity to dusting, hygroscopicity, stability, in particular storage stability, color, odor, flowability, propensity to agglomeration, electrostatic charge, light and temperature sensitivity, mechanical stability and redispersability.

Formulation auxiliaries may comprise, e.g., binders, carrier materials, pulverization/flow auxiliaries, and color pigments, biocides, dispersing agents, anti-foaming agents, viscosity-regulating agents, acids, bases, antioxidants, enzyme stabilizers, enzyme inhibitors, adsorbates, fats, fatty acids, oils or mixtures thereof. Such formulation auxiliaries may be used as drying auxiliaries, in particular in formulation and drying processes, such as spray drying, fluidized bed drying and lyophilization.

Examples for binders are carbohydrates, in particular sugars such as monosaccharides, disaccharides, oligo- and polysaccharides, e.g. dextrins, trehalose, glucose, glucose syrup, maltose, saccharose, fructose and lactose; colloidal substances, such as animal proteins, e.g. gellatin, casein, in particular sodium casein, plant proteins, e.g. soy protein, pea protein, bean protein, lupin, zein, wheat protein, maize protein, and rice protein; synthetic polymers, such as polyethylene glycol, polyvinyl alcohol, and, in particular, the Kollidon trademarks of the company BASF, optionally modified biopolymers, such as lignin, chitin, chitosan, polylactide and modified starches, such as octenylsuccinic anhydride (OSA); rubbers, such as gum acacia; cellulose derivatives, such as methyl cellulose, ethyl cellulose, (hydroxyethyl)methyl cellulose (NEMC), (hydroxypropyl)methyl cellulose (HPMC), carboxymethyl cellulose (CMC); flours, such as maize flour, wheat flour, rye flour, barley flour and rice flour.

Examples of carrier materials are carbohydrates, in particular the sugars mentioned above as binders, and starches, e.g. from maize, rice, potato, wheat and cassava; modified starches, such as octenylsuccinic anhydride; cellulose and microcrystalline cellulose; inorganic minerals or clay, e.g. potter's clay, coal, diatomite, silica, talc and kaolin; farine, e.g. semolina, bran, e.g. wheat bran, the flours mentioned above as binders; salts, such as metal salts, in particular alkali metal salts and earth alkali metal salts of organic acids, such as Mg, Ca, Zn, Na, K citrate, acetate, formate and hydrogen formate, inorganic salts, such as Mg, Ca, Zn, Na, K sulfate, carbonate, silicate or phosphate; earth alkali metal oxides such as CaO and MgO; inorganic buffering agents, such as alkali metal hydrogen phosphates, in particular sodium and potassium hydrogen phosphates, e.g. K₂HPO₄, KH₂PO₄ and Na₂HPO₄; as well as generally the adsorbents mentioned in connection with the preparation, according to the present invention, of metabolites having a low melting point and/or an oily consistency.

Examples of pulverizing agents or flow auxiliaries are diatomite, silica, e.g. the Sipernat trademarks of the company Degussa; potter's clay, carbon/coal, talc and kaolin; the starches, modified starches, inorganic salts, salts of organic acids and buffering agents, mentioned above as carrier materials; cellulose and microcrystalline cellulose.

Examples of other additives are color pigments, such as TiO₂; biocides; dispersants; anti-foaming agents; viscosity-regulating agents, inorganic acids, such as phosphoric acid, nitric acid, hydrochloric acid, sulfuric acid; organic acids, such as saturated and unsaturated mono- and dicarboxylic acids, e.g. formic acid, acetic acid, propionic acid, butyric acid, valeric acid, palmitic acid; stearic acid, oxalic acid, malonic acid, succinic acid, glutaric acid, adipic acid, pimelic acid, maleic acid, and fumaric acid; bases, such as alkali metal hydroxides, e.g. NaOH and KOH; antioxidants; enzyme stabilizers; enzyme inhibitors; adsorbates; fats, fatty acids and oils.

The quota of the above mentioned additives and optionally of further additives, such as coating materials, may vary widely, depending on the specific requirements of the used ingredient as well as on the properties of the additives used, as is known to the person skilled in the art and may lie e.g. in the range from 0.1 to 80% by weight, preferably in the range from 5 to 70% by weight and more preferably in the range from 10 to 60% by weight, relative to the overall weight of the finished formulated product or mixture of materials.

Formulation auxiliaries may be added to the fermentation broth prior to, during or after processing, and, in particular, during drying. An addition of formulation auxiliaries, e.g. prior to concentrating the fermentation broth, may in particular be used for improving the processability of the substances or products to be processed. Before the final drying step, the formulation auxiliaries may be added both to the product obtained in solid form and to a solution or suspension containing the same, e.g. directly to the fermentation broth or to the solution or suspension obtained in the course of processing.

In a preferred embodiment the auxiliaries may be mixed e.g. into a suspension obtained by concentration of a fermentation broth; such a suspension may also be added to a carrier material, e.g. by intermixing. Preferably, formulation auxiliaries may be added after drying, e.g. by applying films or coatings/coating layers to dried particles. Both after drying and also after an optional coating step, further auxiliary agents may be added to the product. Particles resulting from the formulation process may be dried by means of the above described drying processes until the desired moisture content has been reached.

Products obtained in solid form, e.g. particles, granulates and extrudates, may be coated with at least one layer or coating, i.e. with at least one further substance layer. Coating may be effected e.g. in mixers or fluidized beds dispersing or “fluidizing” the particles to be coated, on which subsequently a film or coating material is sprayed. The coating material may be in the dry state, e.g. as powder, or be present in the form of a solution, dispersion, emulsion or suspension in a solvent, e.g. water, organic solvents and mixtures thereof, in particular in water. Any solvent will be removed by means known to the person skilled in the art, e.g. evaporation during or after spraying onto the particles. Furthermore, coating materials such as fats may be applied also as a melt.

Coating materials that can be sprayed on in the form of an aqueous dispersion or suspension are known to the person skilled in the art and are described, e.g., in WO 03/059087. Preferably, they comprise polyolefins such as polyethylene, polypropylene, polyethylene waxes, waxes, inorganic and organic salts, Acronals, such as butylacrolate-methylacrolate copolymer, the Styrofan trademarks of the company BASF, e.g. on the basis of styrene and butadiene, and hydrophobic substances as described in WO 03/059086. When applying such materials the solids content of the coating material may be in the range from 0.1 to 20% by weight, preferably in the range from 0.2 to 10% by weight and more preferably in the range from 0.4 to 5% by weight, each relative to the overall weight of the formulated end product.

Coating materials that can be sprayed on in the form of a solution are e.g. polyethylene glycols, cellulose derivatives such as methyl cellulose, hydroxypropylmethyl cellulose and ethyl cellulose, polyvinyl alcohol, proteins such as gellatin, inorganic and organic salts, carbohydrates such as sugars, e.g. glucose, lactose, fructose, saccharose and trehalose; starches and modified starches. When using such materials, the solids content of the coating material may be in the range from 0.1 to 20% by weight, preferably in the range from 0.2 to 10% by weight and more preferably in the range from 0.4 to 5% by weight, relative to the overall weight of the formulated end product.

Coating materials that can be sprayed on as melts are known to the person skilled in the art and described, e.g., in DE 199 29 257 and WO 92/12645. They may comprise polyethyleneglycols, synthetic fats and waxes, e.g. Polygen WE® of the company BASF, natural fats, such as animal fats, e.g. bee wax, and vegetable fats, e.g. candelilla wax, fatty acids, e.g. animal waxes, tallow acid, palmitic acid, stearic acid, trigylcerides, Edenor products, Vegeole products, montan-ester waxes such as Luwax E® of the company BASF. When using such materials, the solids content of the coating material may be in the range of from 1 to 25% by weight, preferably in the range of from 2 to 25% by weight and more preferably in the range of from 3 to 20% by weight, each relative to the overall weight of the formulated end product.

After drying and/or formulation, whole or ground grains, preferably maize, wheat, barley, millet and/or rye may be added to the product or composition.

In a preferred embodiment stable powdery products may be obtained by converting fermentation solutions containing yeasts (e.g. 9.7% solids content) or lactobacilli (e.g. 12.4% solids content) by means of different formulation methods as described herein below. Preferably, the activity of the yeast fermentation broths is in the range of about 10¹² cfu and in the case of the lactobacilli in the range of about 10¹⁰-10¹¹ cfu.

### Spray Drying of Yeasts:

To an 1,000 g aqueous yeast solution (9.7% solids content) 145.5 g trehalose (binder/film former) are added under stirring (in ice bath). After mixing for 15 min, the cooled solution is dried in a Niro Minor laboratory spraying tower with the following specifications: 1 mm 2-component jet/nozzle, pressure 2 bar, Tₒₙ=125° C. and T_{off}=60-63° C., spray time: 130 min (8-9 g/min). Preferably, the yeast content (dry mass) may be, for instance, about 39%. The activity of the spray dried yeasts may be, for example, at 9.4×10⁴ cfu.

### Spray Drying of Lactobacilli:

To an 1,300 g aqueous solution of lactobacilli (12.4% solids content) are added 241.8 g trehalose (binder/film former) under stirring (in ice bath). After mixing for 15 minutes the cooled solution is dried in a Niro Minor laboratory spraying tower having the following specifications: 1 mm 2-component jet/nozzle, pressure 2 bar, Tₒₙ=125° C. and T_{off}=60-63° C., spray time: 180 min (8-9 g/min). Preferably, the lactobacillus content (dry mass) may be, for instance, about 39%. The activity of the spray dried lactobacilli may be, for example, at about 1.05×10¹⁰ cfu. Preferably, the activity may be, e.g. at 9.4×10⁴ cfu.

### Spray Formulation of Yeasts:

220 g aqueous yeast solution (9.7% solids content is diluted with a further 200 g water to obtain a suitable viscosity for subsequent spraying. Then, under strong agitation, 100 g trehalose (binder/film former) and 110 g Purity gum (modified starch) are added/dissolved at 60° C. to/in the aqueous solution. After mixing for 30 min (at 60° C.) the suspension is transferred to a heated autoclave (at 60° C.). The suspension is subsequently sprayed from above in a laboratory spraying tower (1.1 mm nozzle, pressure 20 bar, temperature in the spraying tower is room temperature) and powdered with silica (Sipernat D17, Degussa) sprayed into the tower from below. In a second step, the powder is dried overnight at room temperature with a suction filter (fluid bed), and the major part of the Sipernat D17 is removed. Preferably, the final dry powder comprises 2-4% Sipernat D17 and the yeast content (dry mass) may be, for example, about 8%. The activity of the spray formulated yeasts may be, for example, at about 6.7×10⁴ cfu.

### Further Spray Formulation of Yeasts:

To/in the 400 g aqueous yeast solution (9.7% solids content) 100 g trehalose (binder/film former) and 100 g Purity gum (modified starch) are added/dissolved at room temperature under strong stirring. After mixing for 45 min (at room temperature) the suspension is transferred to a heated autoclave (at 60° C.). Subsequently, the suspension is sprayed in a laboratory spraying tower from above (1.1 mm nozzle, pressure 20 bar, temperature in the tower is room temperature) and, powdered with silica (Sipernat D17, Degussa), sprayed from below into the tower. In a second step, the powder is dried overnight at room temperature with a suction filter (fluid bed) and the major portion of the Sipernat D17 is removed. Preferably, the final dry powder comprises 2-4% Sipernat D17 and the yeast content (dry mass) may be, for example, about 16%. The activity of the spray formulated yeasts may be, for example, at about 4.1×10⁸ cfu.

### Spray Formulation of Lactobacilli:

To/in a 400 g aqueous solution of lactobacilli (12.4% solids content) are added/dissolved 100 g trehalose (binder/film former) and 100 g Purity gum (modified starch) under strong stirring at room temperature. After mixing for 45 min (at room temperature) the suspension is transferred to an unheated autoclave. The suspension is subsequently sprayed from above in a laboratory spraying tower (1.1 mm nozzle, pressure 30 bar, temperature in the tower is room temperature) and, powdered with silica (Sipernat D17, Degussa), sprayed from below into the tower. In the/a second step, the power was dried overnight at room temperature with a suction filter (fluid bed) and the major part of the Sipernat D17 is removed. Preferably, the final dry powder comprises 2-4% Sipernat D17 and the lactobacillus content (dry mass) may be, for example, about 19%. The activity of the spray formulated lactobacilli may be, for example, at about 4.6×10¹⁰ cfu.

### Further Spray Formulation of Lactobacilli:

400 g aqueous lactobacillus solution (12.4% solids content) are transferred to an unheated autoclave. In a laboratory spraying tower the suspension is subsequently sprayed from above (1.1 mm nozzle, pressure 30 bar, temperature in the tower is room temperature) and, powdered with silica (Sipernat D17, Degussa), sprayed from below into the tower. In a second step, the powder is dried overnight at room temperature with a suction filter (fluid bed) and the major part of the Sipernat D17 is removed. Preferably, the final dry powder comprises 2-4% Sipernat D17 and the lactobacillus content (dry mass) may be, for example, about 95%. The activity of the spray formulated lactobacilli may be, for example, at about 5.4×10¹⁰ cfu.

Lödige Mixer and Extrusion of Yeasts

Into a Lödige mixer with chopper knife are introduced 995 g (dry mass 886 g) maize starch (binder). 14 g PVA (Erkol 5/88, 87-89% degree of hydrolysis) are dissolved in 70 g water and then mixed with 400 g aqueous yeast solution (9.7% solids content). The entire yeast/PVA solution is introduced into the Lödige mixer together with the maize starch in 1-2 min at 25-28° C. (100-350 rpm). The finished mass from the Lödige mixer is then extruded (matrix: 0.7 mm, maximum temperature 43° C.). In the last step, the product (extrudate) is dried in a rotator dryer (MP1), e.g. under the following parameters:

start (t=0 min): product temperature: 21° C., supply air temperature: 26° C., air quantity 600 m³/h,
t=13 min: product temp.: 42° C., supply air temp.: 70° C., air quantity 600 m³/h
end (t=60 min): product temp.: 41° C., supply air temp.: 44° C., air quantity 450 m³/h
end of cooling (t=90 min): product temp.: 41° C., supply air temp.: 44° C., air quantity 450 m³/h

Preferably, the yeast content (dry mass) may be, for example, about 4%. The activity of the yeasts may be, for example, at about 2.3×10⁷ cfu.

Lödige Mixer, Extrusion and Fat Coating of Yeasts:

500 g of the dried product (extrudate) as described under “Lödige mixer and extrusion of yeasts”, supra, are coated with 89 g hard fat in a fluidized bed. The fat is sprayed on as a melt by a two-component jet/nozzle by means of negative pressure absorption. The following temperature/time parameters may be used:

the net spraying time is 14 min (about 6.3 g/min), product temp. 46-49° C., supply air temp. 47-54° C., fat temp. 81-82° C., and air quantity 30 m³/h. After spraying, the coated product is cooled for a further 14 min and the product temperature at the end is 35° C., supply air 36° C., and air quantity 30 m³/h.

Preferably, the yeast content (dry mass) may be, for example, about 3%. The activity of the yeasts may be, for example, at about 1.4×10⁸ cfu.

### Extrusion and Fat Coating of Lactobacilli:

987 g (dry mass 879 g) maize starch (binder) are introduced into a Lödige mixer with chopper knife. 20 g PVA (Erkol 5/88, 87-89% degree of hydrolysis) are dissolved in 70 g water and subsequently mixed with 410 g aqueous lactobacillus solution (12.4% solids content). The entire lactobacillus/PVA solution is introduced into the Lödige mixer containing the maize starch within 1-2 min at 25-28° C. (100-350 rpm). The finished mass is then extruded from the Lödige mixer (matrix: 0.7 mm, maximum temperature 43° C.). In the last step, the product (extrudate) is dried in a rotator dryer (MP1), e.g. under the following parameters:

start (t=0 min): product temperature: 21° C., supply air temperature: 26° C., air quantity 350 m³/h
t=13 min: product temp.: 45° C., supply air temp.: 72° C., air quantity 300 m³/h
end (t=60 min): product temp.: 39° C., supply air temp.: 41° C., air quantity 300 m³/h
end of cooling (t=90 min): product temp.: 32° C., supply air temp.: 33° C., air quantity 300 m³/h

500 g of the dried product/(extrudate) are coated with 89 g hard fed in a fluidized bed. The fat is then sprayed on as melt with a two-component jet/nozzle by means of vacuum absorption. The following temperature/time parameters may be used:

the net spraying time is 11 min (about 8 g/min), product temp. 46-49° C., supply air temp. 47-54° C., fat temp. 81-82° C., and air quantity 30 m³/h. After spraying on the coated product is cooled for 42 min and at the end the product temp. is 33° C. and the supply air temp. is 33° C., air quantity 30 m³/h.

Preferably, the lactobacillus content (dry mass) may be, for example, about 3%. The activity of the lactobacilli may be, for example, at about 5.4×10⁹ cfu.

### Spray Solidification of Yeasts:

63 g Tixosil 38X are introduced into a stirring flask. 102 g aqueous yeast solution (9.7% solids content) are added dropwise in 3 min (stirrer: 600 rpm). The resulting adsorbate is then dried for 4 hours on a suction filter in an air flow. A mixture of 50 g Edenor (hard fat) and 5 g Delios oil (80° C.) are given into a beaker and stirred. 32 g dried absorbate are stirred into this mixture. The finished mixture is then added dropwise to water of about 24 C. Finally, the solidified particles are dried.

Preferably, the yeast content (dry mass) may be, for example, about 3%. The activity of the yeasts may be, for example, at about 2×10³ cfu.

### Spray Solidification of Lactobacilli:

63 g Tixosil are placed in a stirring flask. Over 3 min 102 g aqueous lactobacillus solution (12.4% solids content) are added dropwise (stirrer: 600 rpm). The resulting adsorbate is then dried for 2.5 hours with a suction filter in an air stream. A mixture of 55 g Edenor (hard fat) and 5 g Delios oil (80° C.) is placed in a stirring flask and stirred. Subsequently, 30 g dried adsorbate are stirred into this mixture. The final mixture is added dropwise to water of a temperature of about 24° C. Finally, the solidified particles are dried.

Preferably, the lactobacillus content (dry mass) may be, for example, about 3%. The activity of the lactobacilli may be, for example, at about 5.3×10⁴ cfu.

Liquid preparations suitable for oral administration, for example syrups can be prepared, using water, conventional saccharides such as sucrose, sorbitol and fructose, glycols such as polyethylene glycol and propylene glycol, oils such as sesame seed oil, olive oil and soybean oil, antiseptics such as p-hydroxybenzoate ester, preservatives such as p-hydroxybenzoate derivatives, for example p-hydroxybenzoate methyl and sodium benzoate, and other materials such as flavors. Further, preparations suitable for oral administration, for example tablets, powders and granules can be produced, using conventional saccharides such as sucrose, glucose, mannitol, and sorbitol, starch such as potato, wheat and corn, inorganic materials such as calcium carbonate, calcium sulfate, sodium hydrogen carbonate, and sodium chloride, plant powders such as crystal cellulose, licorice powder and gentian powder, excipients such as pinedex, disintegrators such as starch, agar, gelatin powder, crystal cellulose, carmellose sodium, carmellose calcium, calcium carbonate, sodium hydrogen carbonate and sodium alginate, lubricants such as magnesium stearate, talc, hydrogenated vegetable oils, macrogol, and silicone oil, binders such as polyvinyl alcohol, hydroxypropyl cellulose, methyl cellulose, ethyl cellulose, carmellose, gelatin, and starch glue fluid, surfactants such as fatty acid ester, and plasticizers such as glycerin.

In case of ordinary oral administration, the dose of the microorganism or analog or fragment of the present invention could be (in dry weight) as described hereinabove with respect to the cell number or with respect to the mass, for example, 1 μg to 50 g, 1 μg to 10 g, 1 μg to 5 mg, 1 μg to 1 mg, 0.1 mg to 10 g, 1 mg to 1 g or any other weight per subject per day or in several portions daily. In a preferred embodiment, the subject is a non-human animal. Preferably, the dose is 1 mg to 1 g per 1 kg body weight, more preferably per 1 kg body weight once daily or in several portions daily. The dose may vary depending on the age and species of an subject and the degree of manure odor produced However, these doses and the number of dosages vary depending on the individual conditions.

In a further aspect the invention relates to pharmaceutical compositions comprising a therapeutically effective amount of a microorganism of the present invention or of a derivative or mutant of the present invention or an inactive form of said microorganism of the present invention as described above and can be formulated in various forms, e.g. in solid, liquid, powder, aqueous, lyophilized form.

The pharmaceutical composition may be administered with a pharmaceutically acceptable carrier to a subject, preferably a non-human animal, as described herein. In a specific embodiment, the term “pharmaceutically acceptable” means approved by a regulatory agency or other generally recognized pharmacopoeia for use in subjects, and more particularly in animals.

The term “carrier” refers to a diluent, adjuvant, excipient, or vehicle with which the therapeutic is administered. Such a carrier is pharmaceutically acceptable, i.e. is non-toxic to a recipient at the dosage and concentration employed. It is preferably isotonic, hypotonic or weakly hypertonic and has a relatively low ionic strength, such as provided by a sucrose solution. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers. Suitable pharmaceutical excipients include starch, glucose, sucrose, gelatin, malt, rice, flour, chalk, silica gel, sodium stearate, glycerol monostearate, talc, sodium ion, dried skim milk, glycerol, propylene, glycol, water, ethanol and the like. The composition, if desired, can also contain minor amounts of wetting or emulsifying agents, or pH buffering agents. These compositions can take the form of, e.g., solutions, suspensions, emulsion, powders, sustained-release formulations and the like. Examples of suitable pharmaceutical carriers are described in “Remington's Pharmaceutical Sciences” by E. W. Martin. Some other examples of substances which can serve as pharmaceutical carriers are sugars, such as glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; calcium carbonate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; cranberry extracts and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tabletting agents, stabilizers, anti-oxidants and preservatives, can also be present. It is also advantageous to administer the active ingredients in encapsulated form, e.g. as cellulose encapsulation, in gelatine, with polyamides, niosomes, wax matrices, with cyclodextrins or liposomally encapsulated.

Generally, the ingredients are supplied either separately or mixed together in unit dosage form, for example, as a dry lyophilised powder or water free concentrate in a hermetically sealed container such as an ampoule or sachette indicating the quantity of active agent.

The pharmaceutical composition of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In vitro or in situ assays, e.g. those described in the Examples, may optionally be employed to help identify optimal dosage ranges. The precise dose to be employed in the formulation will also depend on the route of administration, and the seriousness of the symptoms, disease or disorder, and should be decided according to the judgment of the practitioner and each patient's circumstances. The oral route of administration is preferred. Effective doses may be extrapolated from dose-response curves derived from in vitro or (animal) model test systems. Preferably, the pharmaceutical composition is administered directly or in combination with an adjuvant. Adjuvants may be selected from the group consisting of a chloroquine, protic polar compounds, such as propylene glycol, polyethylene glycol, glycerol, EtOH, 1-methyl L-2-pyrrolidone or their derivatives, or aprotic polar compounds such as dimethylsulfoxide (DMSO), diethylsulfoxide, di-n-propylsulfoxide, dimethylsulfone, sulfolane, dimethylformamide, dimethylacetamide, tetramethylurea, acetonitrile or their derivatives. These compounds are added in conditions respecting pH limitations. The composition of the present invention can be administered to an animal. “Animal” as used herein is intended to have the same meaning as commonly understood by one of ordinary skill in the art. Particularly, “animal” encompasses “vertebrates” and more particular mammals, preferably non-human mammals.

The term “administered” means administration of a therapeutically effective dose of the aforementioned composition. By “therapeutically effective amount” is meant a dose that produces the effects for which it is administered, preferably this effect is the reduction of generation of feces odor. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

The methods are preferably applicable to veterinary therapy. The compounds described herein having the desired therapeutic activity may be administered in a physiologically acceptable carrier to a subject, as described herein. Depending upon the manner of administration, the compounds may be formulated in a variety of ways as discussed below. The concentration of the therapeutically active compound in the formulation may vary from about 0.01-100 wt %. The agent may be administered alone or in combination with other treatments.

The administration of the pharmaceutical composition can be done in a variety of ways. The preferable route of administering is the oral route.

The attending veterinary and clinical factors will determine the dosage regimen. As is well known in the medical arts, dosages for any one subject depends upon many factors, including the subject's size, body surface area, age, the particular compound to be administered, sex, time and route of administration, general health, and other drugs being administered concurrently. A typical dose can be, for example, in the range of 0.001 to 1000 however, doses below or above this exemplary range are envisioned, especially considering the aforementioned factors.

The dosages are preferably given once a week, more preferably 2 times, 3 times, 4 times, 5 times or 6 times a week and most preferably daily and even more preferably, 2 times a day or more often. However, during progression of the treatment the dosages can be given in much longer time intervals and in need can be given in much shorter time intervals, e.g., several times a day. In a preferred case the immune response is monitored using herein described methods and further methods known to those skilled in the art and dosages are optimized, e.g., in time, amount and/or composition. Progress can be monitored by periodic assessment. It is also envisaged that the pharmaceutical compositions are employed in co-therapy approaches, i.e. in co-administration with other medicaments or drugs.

Another preferred composition of the present invention is a food or feed composition comprising a microorganims, mutant or derivative thereof as described in connection with the composition of the present invention, further comprising an orally acceptable carrier or excipient. Preferably, the microorganism, mutant or derivative thereof is a microorganism, mutant or derivative of the present invention.

“Food” or “feed” comprises any latable, palatable and/or drinkable stuff for animals, for example, mammals, e.g., productive livestock. An “orally acceptable carrier” is described herein above and is preferably not toxic and of food and/or feed grade. Yet, this term also encompasses the carriers mentioned in connection with the pharmaceutical composition of the present invention. A preferred food or feed composition of the present invention comprises, for example, ground grains, preferably maize, wheat, barley, millet and/or rye.

Another preferred embodiment of the invention is the use of a microorganism, mutant or derivative thereof as described herein above for suppressing feces odor. The term “suppressing feces odor” means that at least the amount of a sulphide compound, methyl mercaptan, cadaverine, putrescine, indole or skatole is decreased when a microorganism according to the present invention is used in comparison to a situation in which a microorganism which is not able to reduce the generation of feces odor is utilized.

Preferably, a microorganism, mutant or derivative thereof as described herein above for suppressing feces odor may be utilized to suppress feces odor in sewage plants, in sewage filtration processes, or in sludge obtained during sewage filtration processes. For instance, a microorganism according to the present invention may be used to suppress feces odor in separated precipitation sludge, in dried or semi dried sludge cakes or during sewage water filtration processes. The microorganisms may be added to the sludge or the liquids present during the filtration process in any suitable form and amount known to the skilled person, preferably as compositions, sprays, mixtures etc. as described herein above.

A further preferred embodiment of the invention is a method for the production of a food or feed composition wherein the method comprises the step of adding a microorganism or derivative or mutant thereof, which are disclosed herein above, to a foodstuff or feedstuff, in particular, the ingredients contained in a foodstuff or feedstuff. These ingredients are known to the person skilled in the art.

In accordance with the present invention, the term “foodstuff and feedstuff” encompasses all eatable and drinkable food and drinks. Accordingly, the microorganism derivative or mutant thereof may be included in a food or drink.

Such food drink or feed can be produced by any general method for producing foods and drinks or feeds known to the person skilled in the art, including adding the active ingredient to a raw or cooked material of the food, drink or feed. The food, drink or feed in accordance with the present invention can be molded and granulated in the same manner as generally used for foods, drinks or feeds. The molding and granulating method includes granulation methods such as fluid layer granulation, agitation granulation, extrusion granulation, rolling granulation, gas stream granulation, compaction molding granulation, cracking granulation, spray granulation, and injection granulation, coating methods such as pan coating, fluid layer coating, and dry coating, puff dry, excess steam method, foam mat method, expansion methods such as microwave incubation method, and extrusion methods with extrusion granulation machines and extruders.

The food, drink or feed according to the present invention includes foods, drinks or feeds comprising the active ingredient. The food, drink or feed to be used in the present invention includes any food, drink or feed. The active ingredient in the food, drink or feed is not specifically limited to any concentration as long as the resulting food, drink or feed can exert its activity of reducing the generation of feces odor. The concentration of the active ingredient is preferably 0.001 to 100% by weight, more preferably 0.01 to 100% by weight and most preferably 0.1 to 100% by weight of the food, drink or feed comprising such active ingredient or with respect to the cell number those described herein.

In a further preferred embodiment, the invention relates to the use of a microorganism, mutant or derivative thereof as described herein above in the preparation of foodstuff or feedstuff. The term “foodstuff” and “feedstuff” have been described herein above and encompasses all eatable and drinkable food and drinks.

In addition, the present invention relates to an additive for food, feed or drinks, which, due to the presence of a microorganism or derivative or mutant thereof as described in connection with the composition of the present invention is, inter alia, capable of reducing the generation of feces odor. Preferably, the microorganism, mutant, or derivative thereof is a microorganism, mutant or derivative of the present invention.

The additive for foods can be produced by a general method for producing additives for foods, drinks or feeds. If necessary, additives for general use in foods, drinks or feeds, for example, additives described in Food Additive Handbook (The Japan Food Additives Association; issued on Jan. 6, 1997) may be added satisfactorily, including sweeteners, colorants, preservatives, thickeners and stabilizers, anti-oxidants, color fixing agents, bleaches, antiseptics, gum base, bitters, enzymes, brightening agents, acidifier, seasonings, emulsifiers, enhancers, agents for manufacture, flavors, and spice extracts. Further, conventional saccharides, starch, inorganic materials, plant powders, excipients, disintegrators, lubricants, binders, surfactants, and plasticizers mentioned previously for pharmaceutical tablets may be added satisfactorily.

The sweeteners include aspartame, licorice, stevia, xylose and rakanka (Momordica grosvenori fruit). The colorants include carotenoid and turmeric oleoresin, flavonold, caramel color, spirulina color, chlorophyll, purple sweet potato color, purple yam color, perilla color, and blueberry color.

The preservatives include, for example, sodium sulfite, benzoates, benzoin extract, sorbates, and propionates. The thickeners and stabilizers include, for example, gums such as gum arable and xanthan gum, alginates, chitin, chitosan, aloe extract, guar gum, hydroxypropyl cellulose, sodium casein, corn starch, carboxymethyl cellulose, gelatin, agar, dextrin, methyl cellulose, polyvinyl alcohol, microfiber cellulose, microcrystalline cellulose, seaweed cellulose, sodium polyacrylate, sodium polyphosphate, carrageenan or yeast cell wall.

The anti-oxidants include, for example, vitamin C group, sodium ethylenediaminetetraacetate, calcium ethylenediaminetetraacetate, erythorbic acid, oryzanol, catechin, quercetin, clove extract, enzyme-treated rutin, apple extract, sesame seed extract, dibutylhydroxytoluene, fennel extract, horseradish extract, water celery extract, tea extract, tocopherols, rapeseed extract, coffee bean extract, sunflower seed extract, ferulio acid, butylhydroxyanisole, blueberry leaf extract, propolis extract, pepper extract, garden balsam extract, gallic acid, eucalyptus extract, and rosemary extract.

The color fixing agents include, for example, sodium nitrite. The bleaches include, for example, sodium sulfite.

The antiseptics include, for example, o-phenyl phenol. The gum base includes, for example, acetylricinoleate methyl, urushi wax, ester gum, elemi resin, urucury wax, kaurigum, carnaubawax, glycerin fatty acid ester, spermaceti wax, copaibabalsam, copal resin, rubber, rice bran wax, cane wax, shellac, jelutong, sucrose fatty acid ester, depolymerized natural rubber, paraffin wax, fir balsam, propylene glycol fatty acid ester, powdered pulp, powdered rice hulls, jojoba oil, polyisobutylene, polybutene, microcrystalline wax, mastic gum, bees wax and calcium phosphate.

The bitters include, for example, iso-alpha-bitter acid, caffeine, kawaratake (Coriolus versieolor) extract, redbark cinchona extract, Phellodendron bark extract, gentian root extract, spice extracts, enzymatically modified naringin, Jamaica cassia extract, theabromine, naringin, cassia extract, absinth extract, isodonis extract, olive tea, bitter orange (Citrus aurantium) extract, hop extract and wormwood extract.

The enzymes include, for example, amylase, trypsin or rennet.

The brightening agents include, for example, urushi wax and japan wax. The acidifier include, for example, adipic acid, itacania acid, citric acids, succinic acids, sodium acetate, tartaric acids, carbon dioxide, lactic acid, phytic acid, fumario acid, malic acid and phosphoric acid. The seasonings include, for example, amino acids such as asparagine, aspartic acid, glutamic acid, glutamine, alanine, isoleucine, glycine, serine, cystine, tyrosine, leucine, and praline, nucleic acids such as sodium inosinate, sodium uridinate, sodium guanylate, sodium cytidylate, calcium ribonucleotide and sodium ribonucleotide, organic acids such as citric acid and succinic acid, potassium chloride, sodium chloride-decreased brine, crude potassium chloride, whey salt, tripotassium phosphate, dipotassium hydrogen phosphate, potassium dihydrogen phosphate, disodium hydrogen phosphate, sodium dihydrogen phosphate, trisodium phosphate and chiorella extract.

The enhancers include, for example, zinc salts, vitamin C group, various amino acids, 5-adenylic acid, iron chloride, hesperidin, various calcined calcium, various non-calcined calcium, dibenzoylthiamine, calcium hydroxide, calcium carbonate, thiamine hydrochloride salt, Dunallella. Oarotene, tocopherol, nicotinic acid, carrot carotene, palm oil carotene, calcium pantothenate, vitamin A, hydroxyproline, calcium dihydrogen pyrophosphate, ferrous pyrophosphate, ferric pyrophosphate, ferritin, heme iron, menaquinone, folic acid and riboflavine.

The agents for manufacture include, for example, processing auxiliaries such as acetone and ion exchange resin. The flavors include, for example, vanilla essence and the spice extracts include, for example, capsicum extract.

These various additives can be added to the active ingredient, taking into consideration the mode of administration, in accordance with the present invention.

It is to be understood that this invention is not limited to the particular methodology, protocols, bacteria, yeasts and reagents etc. described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It must be noted that as used herein and in the appended claims, the singular forms “a”, “an”, and “the”, include plural referents unless the context clearly indicates otherwise. Thus, for example, reference to “a reagent” includes one or more of such different reagents, and reference to “the method” includes reference to equivalent steps and methods known to those of ordinary skill in the art that could be modified or substituted for the methods described herein.

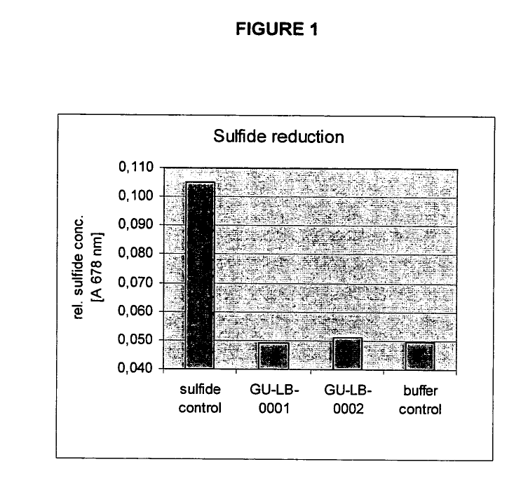

The invention is illustrated by FIGS. 1 to 9 as described in the following:

FIG. 1 shows the results of the colorimetric detection of sulphide after incubation with specific Lactobacillus strains. The reduction is indicated in terms of relative sulphide concentration.

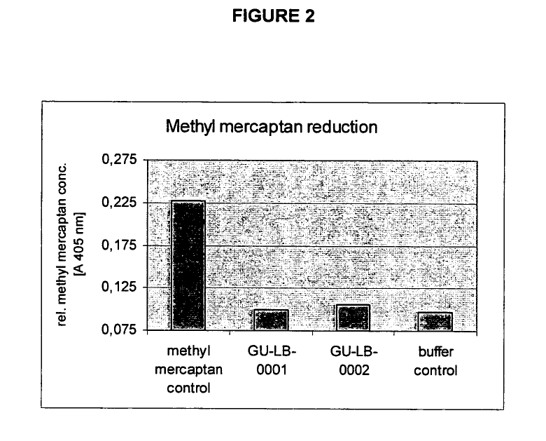

FIG. 2 shows the results of the colorimetric detection of methyl mercaptan after incubation with specific Lactobacillus strains. The reduction is indicated in terms of relative methyl mercaptan concentration.

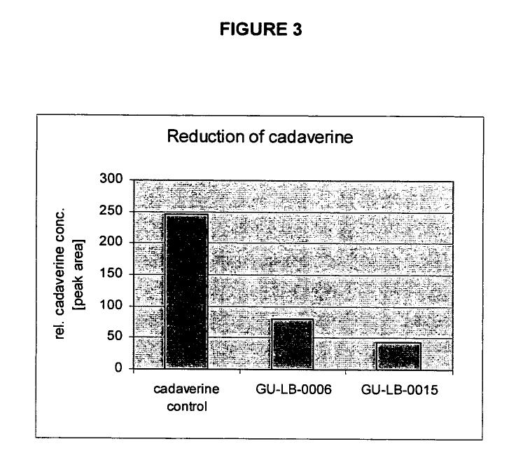

FIG. 3 shows the results of the detection of cadaverine after the incubation with specific Lactobacillus strains (GU-Lb-0006 and GU-Lb-0015). The reduction is indicated in terms of relative cadaverine concentration.

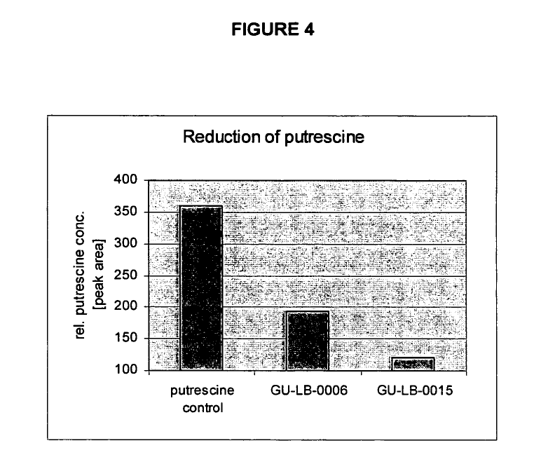

FIG. 4 shows the results of the detection of putrescine after the incubation with specific Lactobacillus strains (GU-Lb-0006 and GU-Lb-0015). The reduction is indicated in terms of relative putrescine concentration.

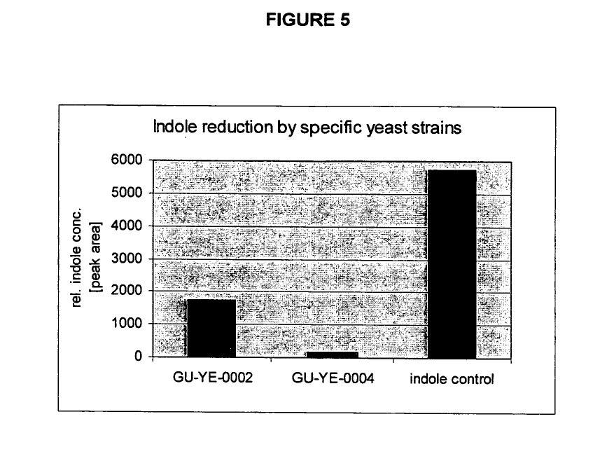

FIG. 5 shows the results of the detection of indole after the incubation with specific yeast strains (GU-Ye-0002 and GU-Ye-0004). The reduction is indicated in terms of relative indole concentration.

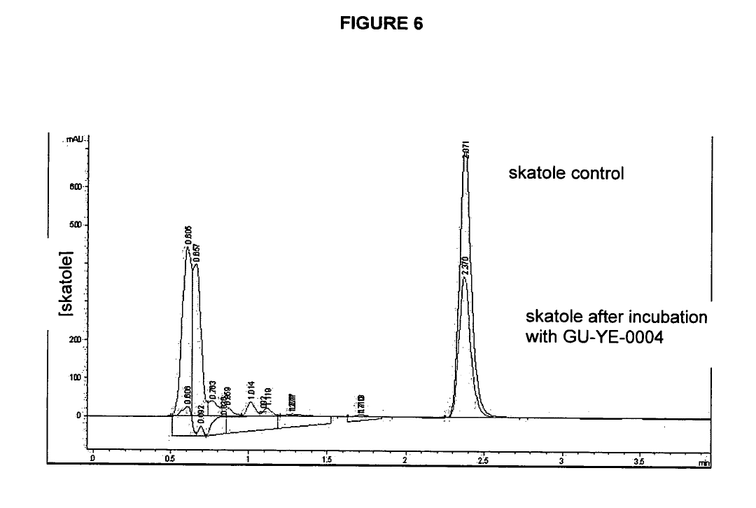

FIG. 6 shows the results of a HPLC analysis of skatole after the incubation with a specific yeast strain (GU-Ye-0004).

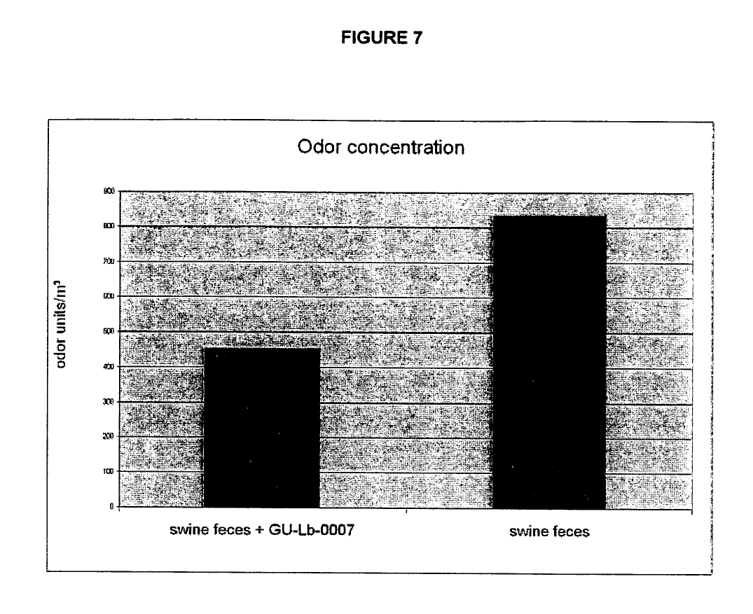

FIG. 7 shows the results of an olfactometrical odor concentration analysis after the ex vivo incubation of pig feces with a specific Lactobacillus strain (GU-Lb-0007).

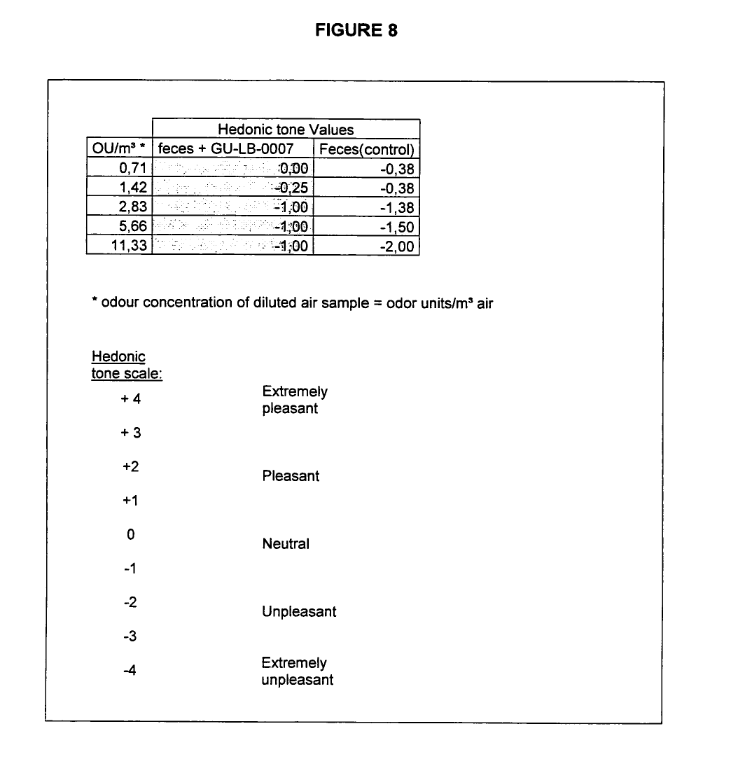

FIG. 8 shows the results of an olfactometrical hedonic tone analysis after the ex vivo incubation of pig feces with a specific Lactobacillus strain (GU-Lb-0007).

A better understanding of the present invention and of its advantages will be obtained from the following examples, which are offered for illustrative purposes only and are not intended to limit the scope of the present invention in any way.

### EXAMPLE 1

### Sulphide Reduction Assay

### Methylene Blue Reaction

Lactic acid bacteria have been identified that are able to reduce sulphides compounds, e.g. hydrogen sulphide. The reduction of hydrogen sulphide was measured as a decrease in sulphide concentration in the presence of a selected lactic acid bacterium.

To identify lactic acid bacteria that are able to reduce hydrogen sulphide the following in vitro assay was performed:

Lactic acid bacteria were anaerobically cultivated by inoculating 10 μl of a freezing culture in 150 μl MRS broth (Difco Manual) and incubation for one day at 37° C. without shaking. The culture was centrifuged for 15 min at 4 000 rpm and the cell pellet was washed one time in 150 μl PBS-buffer (10 mM phosphate, 150 mM NaCl, pH 7.0). The cell pellet afterwards was resuspended in 150 μl oxygen-poor PBS-buffer (PBS buffer freshly boiled and cooled down on ice).

For the assay 50 μl of washed cells of the lactic acid bacterium were mixed with 50 μl of oxygen-poor PBS-buffer and 50 μl sodium sulphide in oxygen-poor aqua dest. (freshly boiled and cooled down on ice) with a sulphide end concentration of 200 μM (48 ppm) in the sample. For a control 50 μl of PBS instead of cells were added. The samples were incubated anaerobically at 37° C. for 1 h, while shaking at 140 rpm. Afterwards cells were centrifuged and the supernatant was derivatised. Therefore 50 μl of the supernatant were added to 50 μl of zinc acetate solution (stock solution: 182 mM zinc acetate in 2% acetic acid; working solution: 1 part stock solution+5 parts aqua dest., freshly boiled and cooled down on ice). Finally 100 μl of DMPD/ferric chloride solution (stock solution: 180 mM DMPD (N,N-Dimethyl-1,4-phenylenediamine sulphate, Sigma), 540 mM FeCl₃, solved in 6 M HCl; working solution: 1 part stock solution+9 parts 6 M HCl) is added. After incubating the sample light-protected for 30 min at room temperature the formation of methylene blue is photometrically measured at a wavelength of 678 nm. The absorption is a measure for sulphide concentration (see FIG. 1).

Media and buffer:

| | |
|---|---|
| MRS-broth | Difco, 150 μl/well |
| PBS-buffer | 10 mM phosphate, 150 mM NaCl, pH 7.0 |
| zinc acetate solution | 182 mM zinc acetate in 2% acetic acid |
| DMPD/ferric | 180 mM DMPD (N,N-Dimethyl-1,4- |
| chloride solution | phenylene-diamine sulphate), 540 mM FeCl₃, |
| | solved in 6M HCl |

### EXAMPLE 2

### Sulphide Reduction Assay

### Olfactoric Assay

Lactic acid bacteria have been identified that are able to reduce sulphides, e.g. hydrogen sulphide. The reduction of hydrogen sulphide was verified by olfactory means of a qualified panel consisting of 5 panellists.

To identify lactic acid bacteria that are able to reduce hydrogen sulphide the following in vitro assay was performed:

Lactic acid bacteria were anaerobically cultivated by inoculating 10 μl of a freezing culture in 150 μl MRS broth (Difco Manual) and incubation for one day at 37° C. without shaking. The culture was centrifuged for 15 min at 4 000 rpm and the cell pellet was washed one time in 150 μl PBS buffer (10 mM phosphate, 150 mM NaCl, pH 7.0). The cell pellet afterwards was resuspended in 150 μl oxygen-poor PBS buffer (PBS buffer freshly boiled and cooled down on ice).

For the assay either 50 μl of washed cells of the lactic acid bacterium or as a control 50 μl of oxygen-poor PBS-buffer were added to 100 μl sodium sulphide in oxygen-poor aqua dest. (freshly boiled and cooled down on ice) with a sulphide end concentration of 500 μM (120 ppm) in the sample.

The samples were incubated anaerobically at 37° C. for 1 h, while shaking at 140 rpm. Afterwards samples were compared to the control without cells by sniffing with the nose. The odor strength was described by the panellists with numbers from 0 to 4, where 0=no odor, 1=very faint odor, 2=faint odor, 3=distinct odor and 4=strong odor. For evaluation the values were averaged. While the control released a strong hydrogen sulphide odor (4), samples with lactic acid bacteria reducing the sulphide concentration exhibited no hydrogen sulphide odor (0).

Media and buffer:

| | |
|---|---|
| MRS-broth | Difco, 150 μl/well |
| PBS-buffer | 10 mM phosphate, 150 mM NaCl, pH 7.0 |

### EXAMPLE 3

### Mercaptan Reduction Assay

Lactic acid bacteria have been identified that are able to reduce mercaptans, e.g. methyl mercaptan. The reduction of methyl mercaptan was measured as a decrease in methyl mercaptan concentration in the presence of a selected lactic acid bacterium.

To identify lactic acid bacteria that are able to reduce methyl mercaptan the following in vitro assay was performed:

Lactic acid bacteria were anaerobically cultivated by inoculating 10 μl of a freezing culture in 150 μl MRS broth (Difco Manual) and incubation for one day at 37° C. without shaking. The culture was centrifuged for 15 min at 4 000 rpm and the cell pellet was washed one time in 150 μl phosphate buffer (50 mM sodium phosphate, pH 8.0). The cell pellet afterwards was resuspended in 150 μl phosphate buffer.

For the assay either 50 μl of washed cells of the lactic acid bacterium or as a control 50 μl of phosphate buffer were mixed with 100 μl of methyl mercaptan in phosphate/DMSO solution (10% DMSO (Dimethyl sulfoxide, Merck) in phosphate buffer) with an end concentration of 500 μM methyl mercaptan (48000 ppm) in the sample. The samples were incubated anaerobically at 37° C. for 1 h, shaking at 140 rpm. Afterwards cells were centrifuged and the supernatant was derivatised. Therefore 20 μl of the supernatant were added to 180 μl of DTNB solution (stock solution: 5 mM DTNB (5,5″-Dithiobis(2-nitrobenzoic acid), Sigma) in phosphate buffer; working solution: 1 part stock solution+19 parts phosphate buffer). After incubating the sample light-protected for 30 min at room temperature the formation of a yellow reduction product is photometrically measured at a wavelength of 405 nm. The adsorption at 405 nm is a measure for methyl mercaptan concentration (see FIG. 2).

Media and buffer:

| | |
|---|---|
| MRS-broth | Difco, 150 μl/well |
| PBS-buffer | 10 mM phosphate, 150 mM NaCl, pH 7.0 |
| Methyl mercaptan/ | 10% DMSO (Dimethyl sulfoxide, Merck) in |
| DMSO solution | phosphate buffer; 500 μM methyl mercaptan |
| DTNB solution | 5 mM DTNB (5,5′-Dithiobis(2-nitrobenzoic |
| | acid), Sigma) in phosphate buffer; |

### EXAMPLE 4

### Mercaptan Reduction Assay

### Olfactoric Assay

Lactic acid bacteria have been identified that are able to reduce mercaptan, e.g. methyl mercaptan. The reduction of methyl mercaptan was verified by olfactory means of a qualified panel consisting of 5 panellists.

To identify lactic acid bacteria that are able to reduce methyl mercaptan the following in vitro assay was performed:

Lactic acid bacteria were anaerobically cultivated by inoculating 10 μl of a freezing culture in 150 μl MRS broth (Difco Manual) and incubation for one day at 37° C. without shaking. The culture was centrifuged for 15 min at 4 000 rpm and the cell pellet was washed one time in 150 μl phosphate buffer (50 mM sodium phosphate, pH 8.0). The cell pellet afterwards was resuspended in 150 μl phosphate buffer.

For the assay either 50 μl of washed cells of the lactic acid bacterium or as a control 50 μl of phosphate buffer were mixed with 100 μl of methyl mercaptan in phosphate/DMSO solution (10% DMSO (Dimethyl sulfoxide, Merck) in phosphate buffer) with an end concentration of 500 μM methyl mercaptan in the sample. The samples were incubated anaerobically at 37° C. for at least 1 h, shaking at 140 rpm. Afterwards samples were compared to the control without cells by sniffing with the nose. The odor strength was described by the panellists with numbers from 0 to 4, where 0=no odor, 1=very faint odor, 2=faint odor, 3=distinct odor and 4=strong odor. For evaluation the values were averaged. While the control released a strong methyl mercaptan odor (4), samples with lactic acid bacteria reducing the methyl mercaptan concentration exhibited less methyl mercaptan odor ranging from 0 to 2.

Media and buffer:

| | |
|---|---|
| MRS-broth | Difco, 150 μl/well |
| Phosphate buffer | 50 mM sodium phosphate, pH 8.0 |
| Methyl mercaptan/ | 10% DMSO (Dimethyl sulfoxide, Merck) in |
| DMSO solution | phosphate buffer; 500 μM methyl mercaptan |

### EXAMPLE 5

### Biogenic Amine Reduction Assay

Lactic acid bacteria have been identified that are able to reduce biogenic amines, e.g. cadaverine or putrescine. The reduction of the biogenic amine was measured as a decrease in amine concentration in the presence of a selected lactic acid bacterium.

To identify lactic acid bacteria that are able to reduce cadaverine or putrescine the following in vitro assay was performed:

Lactic acid bacteria were anaerobically cultivated by inoculating 10 μl of a freezing culture in 150 μl MRS broth (Difco Manual) and incubation for one day at 37° C. without shaking. The culture was centrifuged for 15 min at 4 000 rpm and the cell pellet was washed one time in 150 μl PBS-buffer. The cell pellet afterwards was resuspended in 150 μl PBS-buffer.

For the assay 50 μl of washed cells of the lactic acid bacterium were mixed with 50 μl of PBS-buffer and 50 μl cadaverine or putrescine in PBS-buffer with an amine end concentration of 50 μM in the sample. For a control 50 μl of PBS instead of cells were added. The samples were incubated anaerobically at 37° C. for 1 h, while shaking at 140 rpm. Afterwards cells were centrifuged and the supernatant was derivatised.

Therefore 120 μl of the supernatant were mixed with 40 μl freshly prepared NBD-Chloride solution (2 mg NBD-CI/ml Ethanol) and 80 μl propyl amine solution (50 μM propyl amine in tetra-borate buffer pH 9.75). After incubating the sample for 1 h at 60° C. it is cooled down to room temperature in an ice bath. The pH of the sample is adjusted to pH 6-pH 7. Finally the sample is analysed by HPLC/FL for the presence and quantity of the amine compound. The quantity of cadaverine or putrescine was observed by HPLC analysis, performed on an Agilent chemstation with a Supelco Ascentis™ RP-AMIDE column (15 cm×3 mm, 5 μm). The solvent gradient was as follows: 0 min: 15% acetonitrile/85% citrate buffer pH 3.0, 3 min: 20% acetonitrile/80% citrate buffer pH 3.0, 11 min: 85% acetonitrile/15% citrate buffer pH 3.0, 12 min: 85% acetonitrile/15% citrate buffer pH 3.0, 16 min: 15% acetonitrile/85% citrate buffer pH 3.0, stop after 17 min. The column temperature was 20° C. The constant flow velocity was 1.2 ml/min. Cadaverine or putrescine was identified by Fluorescence analysis (λₑₓ=490 nm, λₑₘ=550 nm) and comparison of retention time to the pure standard substances. The peak area is a measure for amine concentration (see FIGS. 3 and 4).

### EXAMPLE 6

### Growth Monitoring Assay Lactic Acid Bacteria

Lactic acid bacteria have been identified that are able to reduce odorous substances independent of growth. The reduction of the odorous substance was measured as a degree in substance concentration in the presence of a selected lactic acid bacterium.

To identify lactic acid bacteria that are able to reduce odorous substances independent of growth the following in vitro assay was performed: lactic acid bacteria were anaerobically cultivated by inoculating 10 ml of a freezing culture in 150 ml MRS (Difco) and incubation for one day at 37° C. without shaking. The culture was centrifuged for 15 min at 4000 rpm and the cell pallet was washed one time in 150 ml PBS-buffer (10 mM phosphate, 150 mM NaCl, pH 7.0). The cell pellet afterwards was resuspended in 150 ml PBS-buffer. For the assay 50 ml of washed cells of the lactic acid bacterium were mixed with 50 ml of PBS-buffer and 50 ml of the ordeal substance in PBF-buffer. For a growth 50 ml of PBS instead of cells were added. The samples were measured photometrically at 600 nm to determine the optical density. The samples were then incubated anaerobically at 37° C. for one hour, while shaking at 150 rpm. After incubation the optical density at 600 nm was measured again to proof that the cells were not growing. Afterwards cells were centrifuged and the supernatant was derivartised or analyzed directly.

Media and buffer:

| | |
|---|---|
| MRS-broth | Difco, 150 μl/well |
| PBS-buffer | 10 mM phosphate, 150 mM NaCl, pH 7.0 |

### EXAMPLE 7

### Growth Monitoring Assay Yeasts

Yeasts have been identified that are able to reduce odorous substances independent of growth. The reduction of the odorous substance was measured as a decrease in substance concentration in the presence of a selected yeast.

To identify yeasts that are able to reduce odorous substances independent of growth the following in vitro assay was performed:

Yeasts were aerobically cultivated by inoculating 10 μl of a freezing culture in 150 μl YM broth (Difco Manual; 3.0 g yeast extract, 3.0 g malt extract, 5.0 g peptone, 10.0 g dextrose per liter) and incubation for two days at 30° C. shaking. The culture was centrifuged for 15 min at 4 000 rpm and the cell pellet was washed one time in 150 μl PBS-buffer (10 mM phosphate, 150 mM NaCl, pH 7.0). The cell pellet afterwards was resuspended in 150 μl PBS-buffer.

For the assay 50 μl of washed cells of the yeast were mixed with 50 μl of PBS-buffer and 50 μl of the odorous substance in PBS-buffer. For a control 50 μl of PBS instead of cells were added. The samples were measured photometrically at 600 nm to determine the optical density. The samples were then incubated anaerobically at 37° C. for 1 h, while shaking at 140 rpm. After incubation the optical density at 600 nm was measured again to prove that the cells were not growing. Afterwards cells were centrifuged and the supernatant was derivatised or analysed directly.

### EXAMPLE 8

### Growth Monitoring Assay in the Reduction of Indole/Skatol by Yeasts

Yeasts have been identified that are able to reduce indolic compounds, e.g. indole or skatole, independent of growth. The reduction of indole or skatole was measured as a decrease in indole or skatole concentration in the presence of a selected yeast.

To identify yeasts that are able to reduce indole or skatole independent of growth the following in vitro assay was performed:

Yeasts were aerobically cultivated by inoculating 10 μl of a freezing culture in 150 μl YM broth (Difco Manual; 3.0 g yeast extract, 3.0 g malt extract, 5.0 g peptone, 10.0 g dextrose per liter) and incubation for two days at 30° C. shaking. The culture was centrifuged for 15 min at 4 000 rpm and the cell pellet was washed one time in 150 μl PBS-buffer (10 mM phosphate, 150 mM NaCl, pH 7.0). The cell pellet afterwards was resuspended in 150 μl PBS-buffer.

For the assay 50 μl of washed cells of the yeast were mixed with 50 μl of PBS-buffer and 50 μl indole or skatole in PBS-buffer with an indole or skatole end concentration of 200 μM in the sample. For a control 50 μl of PBS instead of cells were added. The samples were measured photometrically at 600 nm to determine the optical density. The samples were then incubated anaerobically at 37° C. for 1 h, while shaking at 140 rpm. After incubation the optical density at 600 nm was measured again to prove that the cells were not growing. Afterwards cells were centrifuged and the supernatant was analysed by HPLC/DAD for the presence and quantity of the indolic compound. The quantity of indole or skatole was observed by HPLC analysis, performed on an Agilent chemstation with an Agilent Zorbax Eclipse XDB-C8 column (3.0×150 mm, 5 μm). The isocratic program was 40% 0.1 M sodium acetate/45% acetonitrile/15% methanol pH 7.2 for 4 min. The column temperature was 25° C. The constant flow velocity was 1 ml/min. Indole or skatole was identified by DAD analysis (λ=220 nm) and comparison of retention time to the pure standard substances. The peak area is a measure for indole or skatole concentration.

### EXAMPLE 9

### Indole Reduction Assay

Yeasts have been identified that are able to reduce indolic compounds, e.g. indole or skatole. The reduction of indolic compounds was measured as a decrease in indole or skatole concentration in the presence of a selected yeast.

To identify yeasts that are able to reduce indole or skatole the following in vitro assay was performed:

Yeasts were aerobically cultivated by inoculating 10 μl of a freezing culture in 150 μl YM broth (Difco Manual; 3.0 g yeast extract, 3.0 g malt extract, 5.0 g peptone, 10.0 g dextrose per liter) and incubation for two days at 30° C. shaking. The culture was centrifuged for 15 min at 4 000 rpm and the cell pellet was washed one time in 150 μl PBS-buffer (10 mM phosphate, 150 mM NaCl, pH 7.0). The cell pellet afterwards was resuspended in 150 μl PBS-buffer.

For the assay 50 μl of washed cells of the yeast were mixed with 100 μl of indole or skatole in PBS-buffer with an end concentration of 200 μM indole or skatole in the sample. For a control 50 μl of PBS instead of cells were added. The samples were incubated anaerobically at 37° C. for 16 h, while shaking at 140 rpm. Afterwards cells were centrifuged and the supernatant was analysed by HPLC/DAD for the presence and quantity of the indolic compound. The quantity of indole or skatole was observed by HPLC analysis, performed on an Agilent chemstation with an Agilent Zorbax Eclipse XDB-C8 column (3.0×150 mm, 5 μm). The isocratic program was 40% 0.1 M sodium acetate/45% acetonitrile/15% methanol pH 7.2 for 4 min. The column temperature was 25° C. The constant flow velocity was 1 ml/min. Indole or skatole was identified by DAD analysis (λ=220 nm) and comparison of retention time to the pure standard substances. The peak area is a measure for indole or skatole concentration (see FIGS. 5 and 6).

### EXAMPLE 10

### Indole Reduction Assay

### Olfactoric Assay

Yeasts have been identified that are able to reduce indolic compounds, e.g. indole or skatole. The reduction of indole or skatole was verified by olfactory means of a qualified panel consisting of 5 panellists.

To identify yeasts that are able to reduce indole or skatole the following in vitro assay was performed:

Yeasts were aerobically cultivated by inoculating 10 μl of a freezing culture in 150 μl YM broth (Difco Manual; 3.0 g yeast extract, 3.0 g malt extract, 5.0 g peptone, 10.0 g dextrose per liter) and incubation for two days at 30° C. shaking. The culture was centrifuged for 15 min at 4 000 rpm and the cell pellet was washed one time in 150 μl PBS buffer (10 mM phosphate, 150 mM NaCl, pH 7.0). The cell pellet afterwards was resuspended in 150 μl PBS buffer.

For the assay either 50 μl of washed cells of the yeast or as a control 50 μl of PBS-buffer were added to 100 μl indole or skatole in PBS-buffer with an indole or skatole end concentration of 300 μM in the sample.

The samples were incubated anaerobically at 37° C. for 1 h, while shaking at 140 rpm. Afterwards samples were compared to the control without cells by sniffing with the nose. The odor strength was described by the panellists with numbers from 0 to 4, where 0=no odor, 1=very faint odor, 2=faint odor, 3=distinct odor and 4=strong odor. For evaluation the values were averaged. While the control released a strong indole or skatole odor (4), samples with yeasts reducing the indole or skatole concentration exhibited no indole or skatole odor (0).

### EXAMPLE 11

### Antibiotic Resistance/Sensitivity Assay

Antibiotic resistance/sensitivity tests are important in the evaluation of what antibiotics could be used in therapy of bacterial infectious diseases. Staphylococcus aureus—ATCC 25923, Escherichia coli—ATCC 25922, and Pseudomonas aeruginosa—ATCC 27853 are often used as quality control organisms since they are of known susceptibility to many antibiotics.

The antibiotic sensitivity of bacteria may be regarded as the lowest test concentration of the antibiotic which completely inhibits the growth of the bacteria; i.e., Minimum Inhibitory Concentration or MIC. Antibiotic resistance may be regarded as the absence of a MIC for a specific antibiotic. The MIC may be determined, for example, by a disc method or a agar plate method.

### Disc Method

A standard method of defining the MIC is the disc method, which involves growth of the target bacteria in the presence of various concentrations of the antibiotic of interest. The type of agar used is essential for the validity of the tests results. Often, Iso-Sensitest agar is used. The hardened agar surface receives a suspension of the test bacteria, which is then spread out evenly over the surface of the agar. The intention is to form a lawn of organisms as growth occurs. Also on the agar surface are discs of an absorbent material. A plate is large enough to house six discs. Each disc has been soaked in a known and different concentration of the same or of different antibiotics.

As growth of the bacteria occurs, antibiotic diffuses out from each disc into the agar. If the concentration of the antibiotic is lethal, no growth of the bacteria will occur. Finally, the diffusing antibiotic will be below lethal concentration, so that growth of bacteria can occur. The result is a ring of no growth around a disc. From comparison with known standards, the diameter of the growth inhibition ring will indicate whether the bacteria are sensitive/resistant to the antibiotic.

### Agar Plate Method

The following stock solutions of an antibiotic are prepared with sterile water: 10, 100, and 1000 ug/ml. The calculated volume of the antibiotic stock solution is added to each agar deep previously melted, and cooled to 50° C.; the agar is mixed and poured into the plates.

### Antibiotic Agar Plate Series:

| | | | Volume to be | |
|---|---|---|---|---|
| Plate No. | μg/ml | μg per 20 ml | added in ml | Stock μg/ml |
| 1 | 0 | 0 | — | — |
| 2 | 0.1 | 2.0 | 0.20 | 10.0 |
| 3 | 0.2 | 4.0 | 0.40 | 10.0 |
| 4 | 0.4 | 8.0 | 0.80 | 10.0 |
| 5 | 1.0 | 20.0 | 0.20 | 100.0 |
| 6 | 2.0 | 40.0 | 0.40 | 100.0 |
| 7 | 4.0 | 80.0 | 0.80 | 100.0 |
| 8 | 6.0 | 120.0 | 0.12 | 1000.0 |
| 9 | 8.0 | 160.0 | 0.16 | 1000.0 |
| 10 | 10.0 | 200.0 | 0.20 | 1000.0 |

After the plates have solidified and dried, each plate is divided into eight sectors with a marker on the back of the plate. A dilution of each culture is prepared by adding the overnight broth culture to 1 ml of saline until the turbidity approximately matches that of a McFarland 0.5 nephelometry standard. A sterile cotton-tipped applicator is dipped into the bacterial suspension and the excess fluid is squeezed out against the inside of the tube. Then a single radial streak of an inch in length is made to the corresponding sector of each plate of the series, beginning with the control plate (no antibiotic) and progressing through the increasing concentration plates. After the inocula have dried or have been absorbed into the agar plate medium the plates are closed and incubated for 24 hours at 35° C. Finally growth is observed and recorded using the following scale: growth equivalent to control ++++; moderate growth +++; intermediate growth ++; scant growth +; no growth −. The MIC is the lowest concentration of the antibiotic tested that yields complete inhibition of growth.

### EXAMPLE 12

### Feces Odor Reduction Assay

### Odor Concentration Assay

Lactic acid bacteria and yeasts have been identified that are able to reduce the odor concentration of feces ex vivo. The reduction of odor concentration was measured olfactometrically as an increase of the odor threshold of pig feces in the presence of selected lactic acid bacteria or yeasts.

To identify lactic acid bacteria that are able to reduce the odor concentration of feces the following ex vivo assay was performed:

Lactic acid bacteria were anaerobically cultivated by inoculating 10 μl of a freezing culture in 1 ml MRS broth (Difco Manual) and incubation for one day at 37° C. without shaking. The culture was centrifuged for 15 min at 4 000 rpm and the cell pellet was washed one time in 1 ml PBS buffer (10 mM phosphate, 150 mM NaCl, pH 7.0). The cell pellet afterwards was resuspended in 1 ml PBS buffer.

For the assay 10⁸ cells were given to 50 g fresh pig feces in 1 liter water. The compounds were mixed. For a control 50 g fresh pig feces in 1 liter water were used. Incubation was performed for 3 h at 37° C. in an airtight container without agitation. After incubation air was drawn from the container into inert Nalophan bags.

The assay was carried out with strain GU-Lb-0007.

Analysis of air samples was performed by a professional panel consisting of 8 persons in accordance with the requirements provided in standard EN 13725. Air samples were diluted to different dilution steps by means of an olfactometer and sniffed/tested by the panellists. Panellists react to odor recognition by pressing a button. If the dilution 1:500 of the air sample is recognized as odor by the panellist, the odor concentration of the sample 500 odor units/m3 (OU/m³), if the sample is recognized as odor even if diluted 1:1000, the odor concentration of the sample is 10000 U/m³ (according to standard EN 13725).

As can be derived from FIG. 7 the addition of the Lactobacillus strain GU-Lb-0007 to swine feces reduced the concentration of swine feces odor.

### EXAMPLE 13

### Feces Odor Reduction Assay

### Hedonic Assay

Lactic acid bacteria and yeasts have been identified that are able to improve the hedonic tone of feces ex vivo. The improvement of hedonic tone was measured olfactometrically in the presence of selected lactic acid bacteria or yeasts.

The identify lactic acid bacteria that are able to improve the hedonic tone of feces the following ex vivo assay was performed:

Lactic acid bacteria were anaerobically cultivated by inoculating 10 μl of a freezing culture in 1 ml MRS broth (Difco Manual) and incubation for one day at 37° C. without shaking. The culture was centrifuged for 15 min at 4 000 rpm and the cell pellet was washed one time in 1 ml PBS buffer (10 mM phosphate, 150 mM NaCl, pH 7.0). The cell pellet afterwards was resuspended in 1 ml PBS buffer.

For the assay 10⁸ cells were given to 50 g fresh pig feces in 1 liter water. The compounds were mixed. For a control 50 g fresh pig feces in 1 liter water were used. Incubation was performed for 3 h at 37° C. in an airtight container. After incubation air was drawn from the container into inert Nalophan bags.

The assay was carried out with strain GU-Lb-0007.

Analysis of air samples was performed by a professional panel consisting of 8 persons in accordance with the requirements provided in standard EN 13725. Air samples were diluted to different dilution steps in accordance with the odor concentration of the samples, as assayed in Example 12, by means of an olfactometer following the regulations provided in EN 13725.

The following dilutions were carried out:

Feces plus lactic acid bacteria:

| Odor concentration (OU/m³) | Dilution (factor Z) |
|---|---|
| 181.20 | 2.5 |
| 90.60 | 5 |
| 45.30 | 10 |
| 22.65 | 20 |
| 11.33 | 40 |
| 5.66 | 80 |
| 2.83 | 160 |
| 1.42 | 320 |
| 0.71 | 640 |

Feces alone:

| Odor concentration (OU/m³) | Dilution (factor Z) |
|---|---|
| 13.36 | 62.5 |
| 6.68 | 125 |
| 3.34 | 250 |
| 1.67 | 500 |
| 0.84 | 1000 |
| 0.42 | 2000 |
| 0.21 | 4000 |
| 0.10 | 8000 |
| 0.05 | 16000 |

Subsequently, the samples were sniffed/tested by the panellists. Panellists assign marks to the odor of the air sample. The scale was −4 extremely unpleasant to +4 extremely pleasant. As can be derived from FIG. 8 the addition of the Lactobacillus strain GU-Lb-0007 to swine feces improved the hedonic tone of swine feces odor.

### CITED REFERENCES

Bunton B (2007): Monitoring and Modeling of Emissions from Concentrated Animal Feeding Operations; Overview of Methods. Environ Health Perspect 115(2): 303-307
Burnett W E (1969): Air pollution from animal wastes: determination of malodors by gas chromatographic and organoleptic techniques. Environ Sci Technol 3:744-9
Elli M, Zink R, Rytz A, Reniero R and Morelli L (2000) Iron requirement of Lactobacillus spp. in completely chemically defined growth media. J Appl Microbiol 88:695-703
Fakhoury K J, Heber A J, Shao P and Ni J Q (2000) Correlation of odor detection thresholds with concentrations of hydrogen sulphide, ammonia and trace gases emitted from swine manure. ASEA Meeting Presentation 2000, Milwaukee, Wis.
Geng A L, Chen X G, Gould W D, Ng Y L, Yan R, Lee C C and Liang D T (2004) Removal of odourous sulphur-containing gases by a new isolate from activated sludge. Water Sci Technol 50:291-7
Hobbs P J, Pain B F, Kay R M and Lee P A (1996) Reduction of odorous compounds in fresh pig slurry by dietary control of crude protein. J Sci Food Agric 71:508-514
Houdijk et al., 2002
Hidaka H, Eida T, Takizawa T, Tokanaga T and Tashiro Y (1986) Effect of fructooligosaccharides on intestinal flora and human health. Bifidobact. Microflora 5:37-50
Kim M K, Choi K M, Yin C R, Lee K Y, Im W T, Lim J H and Lee S T (2004) Odourous swine wastewater treatment by purple non-sulfur bacteria, Rhodopseudomonas palustris, isolated from eutrophicated ponds. Biotechnol. Lett. 26:819-22
Mackie R I, Stroot P G and Varel V H (1998) Biochemical identification and biological origin of key odor components in livestock waste. J Anim Sci 76:1331-42
McCrory D F and Hobbs P J (2001) Additives to reduce ammonia and odor emissions from livestock wastes: a review. J Environ Qual 30:345-55
Miner J R (1977) Characterization of odors and other volatile emissions. Agric Environ 3:129-37
Miner J R (1975) Management of odors associated with livestock production. Proc. 3^{rd} Int. Symp. Livest. Waste, ASAE Urbana-Champaign, Ill. pp. 378-380
Miner J R (1995) Nature and control of odors from pork production facilities: A review of literature. National Pork Producers Council, Des Moines, Iowa.
Moeser A J and van Kempen T (2001) Dietary fiber level and xylanase affects nutrient digestibility and waste production in grower pigs. American Society of Animal Science, 472
Moore et al. (1987)
Naidu A S, Xie X, Leumer D A, Harrison S, Burrill M J and Fonda E A (2002) Reduction of sulphide, ammonia compounds, and adhesion properties of Lactobacillus casei strain KE99 in vitro. Current Microbiology 44:196-205
O'Neill D H and Phillips V R (1992) A review of the control of odour nuisance from livestock buildings: Part 3, properties of the odorous substances which have been identified in livestock wastes or in the air around them. J Agric Engng Res 53: 23-50
Portejoie S, Dourmad J Y, Martinez J and Lebreton Y (2004) Effect of lowering dietary crude protein on nitrogen excretion, manure composition and ammonia emission from fattening pigs. Livestock Production Science 91:45-55
Risley C R, Kornegay E T, Lindemann M D, Wood C M and Eigel W N (1992) Effect of feeding organic acids on selected intestinal content measurements at varying times postweaning in pigs. J Anim Sci 70:196-206
Ritter W F (1989) Odour control of livestock wastes: state-of-the-art in north america. J Agric Eng Res 42:51-62
Sato H, Hirose T, Kimura T, Moriyama Y and Nakashima Y (2001) Analysis of malodorous volatile substances of human waste: feces and urine. J Health Sci 47: 483-90
Schaefer J (1977) Sampling, characterisation and analysis of malodours. Agric and Environ 3:121-7
Schaefer (1997)
Suarez F L, Springfield J and Levitt M D (1998) Identification of gases responsible for the odour of human flatus and evaluation of a device purported to reduce this odour. Gut 43:100-4
Sutton A L, Mathew A G, Scheidt A B, Patterson J A and Kelly D T (1991) Effect of carbohydrate source and organic acids on intestinal microflora and performance of the weanling pig. Proc. Fifth Cong. on Digestive Physiol. in Pigs, Wageningen, The Netherlands, pp. 422-427
Sutton A L, Kephart K B, Patterson J A, Mumma R, Kelly D T, Bogus E, Jones D D and Heber A (1996) Manipulating swine diets to reduce ammonia and odor emissions. Proc. 1^{st} Int. Conf. Air Pollution from Agric. Operations, Kansas City, Mo. pp. 445-452
Sutton A L, Kephart K B, Verstegen M W A, Canh T T and Hobbs P J (1999) Potential for reduction of odorous compounds in swine manure through diet modification. J Anim Sci 77:430-9
Tabor C W and Tabor H (1985) Polyamines in microorganisms. Microbiological Reviews March:91-99
van Kempen T (2001) Dietary adipic acid reduces ammonia emissions from swine excreta. J Anim Sci 79:2412-2417
van Kempen T, van Heugten E and Powers W (2003) Impact of diet on odor. North Carolina State University, Department of Animal Science, Iowa State University, Department of Animal Science, Extension Swine Husbandry, Annual Swine Report 2003
Yashuhara A (1987) Identification of volatile compounds in poultry manure by gases chromatography-mass spectrometry. J Chromatogr 387:371-8
Yumoto I, Hirota K, Yamaga S, Nodasaka Y, Kawasaki T, Matsuyama H and Nakajima K (2004) Bacillus asahii sp. nov., a novel bacterium isolated from soil with the ability to deodorize the bad smell generated from short-chain fatty acids. Int J Evol Microbiol 54(Pt):1997-2001
Yun S I and Ohta Y (2005) Removal of volatile fatty acids with immobilized Rhodococcus sp. B261. Bioresour Technol 96:41-6

1. A microorganism which is able to reduce the generation of feces odor by decreasing the amount of at least one compound selected from the group consisting of
(i) a sulphide compound,
(ii) methyl mercaptan;
(iii) cadaverine;
(iv) putrescine;
(v) indole; and
(vi) skatole;
and wherein said decrease in the amount of the at least one compound is independent of the growth of the microorganism.

2. The microorganism of claim 1, which is a microorganism belonging to the genus of Lactobacillus, or a yeast.

3. The microorganism of claim 2, wherein said Lactobacillus is Lactobacillus paracasei ssp. paracasei, Lactobacillus rhamnosus, Lactobacillus acidophilus, Lactobacillus crispatus, Lactobacillus plantarum, Lactobacillus delbrückii ssp. delbrückii or Lactobacillus curvatus.

4. The microorganism of claim 2, wherein the yeast is a yeast belonging to the genus Cryptococcus, Kluyveromyces, Candida or Metschnikowia.

5. The microorganism of claim 4, wherein said Cryptococcus is Cryptococcus laurentii, wherein said Kluyveromyces is Kluyveromyces marxianus, wherein said Candida is Candida haemulonii, or wherein said Metschnikowia is Metschnikowia reukaufii.

6-8. (canceled)

9. The microorganism of claim 1, wherein said decrease is a decrease of the amount of a sulphide compound and wherein the microorganism is selected from the group consisting of Lactobacillus paracasei ssp. paracasei GU-Lb-0001 (DSM 18456), Lactobacillus rhamnosus GU-Lb-0002 (DSM 18457), Lactobacillus rhamnosus GU-Lb-0005 (DSM 18460), Lactobacillus acidophilus GU-Lb-0007 (DSM 18462), Lactobacillus crispatus GU-Lb-0009 (DSM 18464), Lactobacillus delbrückii ssp. delbrückii GU-Lb-0010 (DSM 18465), Lactobacillus plantarum GU-Lb-0013 (DSM 18468), and Lactobacillus acidophilus GU-Lb-0014 (DSM 18469), or a mutant or derivative thereof, wherein said mutant or derivative retains the ability to decrease the amount of a sulphide compound.

10. (canceled)

11. The microorganism of claim 1, wherein said decrease is a decrease of the amount of methyl mercaptan and wherein the microorganism is selected from the group consisting of Lactobacillus paracasei ssp, paracasei GU-Lb-0001 (DSM 18456), Lactobacillus rhamnosus GU-Lb-0002 (DSM 18457), Lactobacillus rhamnosus GU-Lb-0005 (DSM 18460), and Lactobacillus paracasei ssp. paracasei GU-Lb-0008 (DSM 18463), or a mutant or derivative thereof, wherein said mutant or derivative retains the ability to decrease the amount of methyl mercaptan.

12. (canceled)

13. The microorganism of claim 1, wherein said decrease is a decrease of the amount of cadaverine and wherein the microorganism is selected from the group consisting of Lactobacillus acidophilus GU-Lb-0003 (DSM 18458), Lactobacillus acidophilus GU-Lb-0004 (DSM 18459), Lactobacillus acidophilus GU-Lb-0006 (DSM 18461), Lactobacillus curvatus GU-Lb-0011 (DSM 18466), Lactobacillus crispatus GU-Lb-0012 (DSM 18467), Lactobacillus acidophilus GU-Lb-0014 (DSM 18469), Lactobacillus acidophilus GU-Lb-0015 (DSM 18470), Cryptococcus laurentii GU-Ye-0001 (DSM 18471), and Candida haemulonii GU-Ye-0003 (DSM 18473), or a mutant or derivative thereof, wherein said mutant or derivative retains the ability to decrease the amount of cadaverine.

14. (canceled)

15. The microorganism of claim 1, wherein said decrease is a decrease of the amount of putrescine and wherein the microorganism is selected from the group consisting of Lactobacillus acidophilus GU-Lb-0003 (DSM 18458). Lactobacillus acidophilus GU-Lb-0004 (DSM 18459), Lactobacillus acidophilus GU-Lb-0006 cDSM 18461), Lactobacillus curvatus GU-Lb-0011 (DSM 18466), Lactobacillus crispatus GU-Lb-0012 (DSM 18467), Lactobacillus acidophilus GU-Lb-0014 (DSM 18469), Lactobacillus acidophilus GU-Lb-0015 (DSM 18470), Cryptococcus laurentii GU-Ye-0001 (DSM 18471), and Candida haemulonii GU-Ye-0003 (DSM 18473), or a mutant or derivative thereof, wherein said mutant or derivative retains the ability to decrease the amount of putrescine.

16. (canceled)

17. The microorganism of claim 1, wherein said decrease is a decrease of the amount of indole and wherein the microorganism is selected from the group consisting of Cryptococcus laurentii GU-Ye-0001 (DSM 18471), Kluyveromyces marxianus GU-Ye-0002 (DSM 18472), Candida haemulonii GU-Ye-0003 (DSM 18473), and Metschnikowia reukaufii GU-Ye-0004 (DSM 18474), or a mutant or derivative thereof, wherein said mutant or derivative retains the ability to decrease the amount of indole.

18. (canceled)

19. The microorganism of claim 1, wherein said decrease is a decrease of the amount of skatole and wherein the microorganism is selected from the group consisting of Cryptococcus laurentii GU-Ye-0001 (DSM 18471), Kluyveromyces marxianus GU-Ye-0002 (DSM 18472), Candida haemulonii GU-Ye-0003 (DSM 18473), and Metschnikowia reukaufii GU-Ye-0004 (DSM 18474), or a mutant or derivative thereof, wherein said mutant or derivative retains the ability to decrease the amount of skatole.

20. (canceled)

21. The microorganism of claim 1, wherein said decrease is a simultaneous decrease of the amount of
(a) a sulphide compound and methyl mercaptan;
(b) cadaverine and putrescine;
(c) cadaverine and a sulphide compound;
(d) putrescine and a sulphide compound;
(e) cadaverine, putrescine and a sulphide compound;
(f) indole and skatole;
(g) cadaverine and indole;
(h) cadaverine and skatole;
(i) putrescine and indole;
(i) putrescine and skatole;
(k) indole, skatole and cadaverine;
(l) indole, skatole and putrescine;
(m) indole, cadaverine and putrescine;
(n) skatole, cadaverine and putrescine; or
(o) cadaverine, putrescine, indole and skatole.

22. The microorganism of claim 21, wherein the microorganism which can simultaneously decrease the amount of (a) is selected from the group consisting of Lactobacillus paracasei ssp. paracasei GU-Lb-0001 (DSM 18456), Lactobacillus rhamnosus GU-Lb-0002 (DSM 18457) and Lactobacillus rhamnosus GU-Lb-0005 (DSM 18460), or mutant or derivative thereof, wherein said mutant or derivative retains the ability to simultaneously decrease the amount of a sulphide compound and methyl mercaptan.

23. (canceled)

24. The microorganism of claim 21, wherein the microorganism which can simultaneously decrease the amount of (b), is selected from the group consisting of Lactobacillus acidophilus GU-Lb-0003 (DSM 18458), Lactobacillus acidophilus GU-Lb-0004 (DSM 18459), Lactobacillus acidophilus GU-Lb-0006 (DSM 18461), Lactobacillus curvatus GU-Lb-0011 (DSM 18466), Lactobacillus crispatus GU-Lb-0012 (DSM 18467), Lactobacillus acidophilus GU-Lb-0014 (DSM 18469), Lactobacillus acidophilus GU-Lb-0015 (DSM 18470), Cryptococcus laurentii GU-Ye-0001 (DSM 18471) and Candida haemulonii GU-Ye-0003 (DSM 18473), or a mutant or derivative thereof, wherein said mutant or derivative retains the ability to simultaneously decrease the amount of cadaverine and putrescine.

25. (canceled)

26. The microorganism of claim 21, wherein the microorganism which can simultaneously decrease the amount of (c) is Lactobacillus acidophilus GU-Lb-0014 (DSM 18469), or a mutant or derivative thereof, wherein said mutant or derivative retains the ability to simultaneously decrease the amount of cadaverine and a sulphide compound.

27. (canceled)

28. The microorganism of claim 21, wherein the microorganism which can simultaneously decrease the amount of (d) is Lactobacillus acidophilus GU-Lb-0014 (DSM 18469), or a mutant or derivative thereof, wherein said mutant or derivative retains the ability to simultaneously decrease the amount of putrescine and a sulphide compound.

29. (canceled)

30. The microorganism of claim 21, wherein the microorganism which can simultaneously decrease the amount of (e) is Lactobacillus acidophilus GU-Lb-0014 (DSM 18469), or a mutant or derivative thereof, wherein said mutant or derivative retains the ability to simultaneously decrease the amount of cadaverine, putrescine and a sulphide compound.

31. (canceled)

32. The microorganism of claim 21, wherein the microorganism which can simultaneously decrease the amount of (f) is selected from the group consisting of Cryptococcus laurentii GU-Ye-0001 (DSM 18471), Kluyveromyces marxianus GU-Ye-0002 (DSM 18472), Candida haemulonii GU-Ye-0003 (DSM 18473) and Metschnikowia reukaufii GU-Ye-0004 (DSM 18474), or a mutant or derivative thereof, wherein said mutant or derivative retains the ability to simultaneously decrease the amount of indole and skatole.

33. (canceled)

34. The microorganism of claim 21, wherein the microorganism which can simultaneously decrease the amount of (g) is selected from the group consisting of Cryptococcus laurentii GU-Ye-0001 (DSM 18471) and Candida haemulonii GU-Ye-0003 (DSM 18473), or a mutant or derivative thereof, wherein said mutant or derivative retains the ability to simultaneously decrease the amount of cadaverine and indole.

35. (canceled)

36. The microorganism of claim 21, wherein the microorganism which can simultaneously decrease the amount of (h) is selected from the group consisting of Cryptococcus laurentii GU-Ye-0001 (DSM 18471) and Candida haemulonii GU-Ye-0003 (DSM 18473), or a mutant or derivative thereof, wherein said mutant or derivative retains the ability to simultaneously decrease the amount of cadaverine and skatole.

37. (canceled)

38. The microorganism of claim 21, wherein the microorganism which can simultaneously decrease the amount of (i) is selected from the group consisting of Cryptococcus laurentii GU-Ye-0001 (DSM 18471) and Candida haemulonii GU-Ye-0003 (DSM 18473), or a mutant or derivative thereof, wherein said mutant or derivative retains the ability to simultaneously decrease the amount of putrescine and indole.

39. (canceled)

40. The microorganism of claim 21, wherein the microorganism which can simultaneously decrease the amount of (j) is selected from the group consisting of Cryptococcus laurentii GU-Ye-0001 (DSM 18471) and Candida haemulonii GU-Ye-0003 (DSM 18473), or a mutant or derivative thereof, wherein said mutant or derivative retains the ability to simultaneously decrease the amount of putrescine and skatole.

41. (canceled)

42. The microorganism of claim 21, wherein the microorganism which can simultaneously decrease the amount of (k) is selected from the group consisting of Cryptococcus laurentii GU-Ye-0001 (DSM 18471) and Candida haemulonii GU-Ye-0003 (DSM 18473), or a mutant or derivative thereof, wherein said mutant or derivative retains the ability to simultaneously decrease the amount of indole, skatole and cadaverine.

43. (canceled)

44. The microorganism of claim 21, wherein the microorganism which can simultaneously decrease the amount of (l) is selected from the group consisting of Cryptococcus laurentii GU-Ye-0001 (DSM 18471) and Candida haemulonii GU-Ye-0003 (DSM 18473), or a mutant or derivative thereof, wherein said mutant or derivative retains the ability to simultaneously decrease the amount of indole, skatole and putrescine.

45. (canceled)

46. The microorganism of claim 21, wherein the microorganism which can simultaneously decrease the amount of (m) is selected from the group consisting of Cryptococcus laurentii GU-Ye-0001 (DSM 18471) and Candida haemulonii GU-Ye-0003 (DSM 18473), or a mutant or derivative thereof, wherein said mutant or derivative retains the ability to simultaneously decrease the amount of indole, cadaverine and putrescine.

47. (canceled)

48. The microorganism of claim 21, wherein the microorganism which can simultaneously decrease the amount of (n) is selected from the group consisting of Cryptococcus laurentii GU-Ye-0001 (DSM 18471) and Candida haemulonii GU-Ye-0003 (DSM 18473), or a mutant or derivative thereof, wherein said mutant or derivative retains the ability to simultaneously decrease the amount of skatole, cadaverine and putrescine.

49. (canceled)

50. The microorganism of claim 21, wherein the microorganism which can simultaneously decrease the amount of (o) is selected from the group consisting of Cryptococcus laurentii GU-Ye-0001 (DSM 18471) and Candida haemulonii GU-Ye-0003 (DSM 18473), or a mutant or derivative thereof, wherein said mutant or derivative retains the ability to simultaneously decrease the amount of cadaverine, putrescine, indole and skatole.

51. An inactive form of the microorganism of claim 1, which is able to reduce the generation of feces odor.

52. The inactive form of claim 51, which is thermally inactivated or lyophilized.

53. A composition comprising the microorganism of claim 1 or an inactive form of the microorganism which is able to reduce the generation of feces odor.

54. The composition of claim 53 which is a pharmaceutical composition optionally comprising a pharmaceutically acceptable carrier or excipient.

55. The composition of claim 53 which is a food or feed composition, further comprising an orally acceptable carrier or excipient.

56. A method for suppressing feces odor comprising utilizing the microorganism of claim 1 or an inactive form of the microorganism which is able to reduce the generation of feces odor for suppressing feces odor.

57. (canceled)

58. A method for the production of a food or feed composition comprising adding the microorganism of claim 1 or an inactive form of the microorganism which is able to reduce the generation of feces odor to a foodstuff or feedstuff.

59. An additive for food, feed or drinks comprising the microorganism of claim 1 or an inactive form of the microorganism which is able to reduce the generation of feces odor.
